(19) United States
(12) Patent Application Publication
(10) Pub. No.: US 2016/0166664 A1
(43) Pub. Date: Jun. 16, 2016

(54) COMPOSITIONS AND METHODS FOR INDUCTION OF ANTIGEN-SPECIFIC TOLERANCE

(71) Applicants: Northwestern University, Evanston, IL (US); Myelin Repair Foundation, Inc., Saratoga, CA (US)

(72) Inventors: Stephen Miller, Oak Park, IL (US); Russell L. Bromley, Redwood City, CA (US); Michael A. Pleiss, Sunnyvale, CA (US); Daniel Getts, Chicago, IL (US); Aaron Martin, Chicago, IL (US)

(73) Assignee: Northwestern University, Evanston, IL (US)

(21) Appl. No.: 14/807,397

(22) Filed: Jul. 23, 2015

Related U.S. Application Data
(63) Continuation of application No. 13/144,918, filed on Dec. 5, 2011, now abandoned.
(60) Provisional application No. 61/145,941, filed on Jan. 20, 2009.

(51) Int. Cl. A61K 39/00; A61K 47/48

(52) U.S. Cl. CPC A61K 39/0008; A61K 47/48176; A61K 47/482; A61K 47/48815; A61K 2039/55555; A61K 2039/6093

(57) ABSTRACT

The present invention utilizes carrier particles to present antigen peptides and proteins to the immune system in such a way as to induce antigen specific tolerance. The carrier particle is designed in order to trigger an immune tolerance effect. The invention is useful for treatment of immune related disorders such as autoimmune disease, transplant rejection and allergic reactions.

### CROSS-REFERENCE

This application claims the benefit of U.S. Provisional Application Nos. 61/145,941, filed on Jan. 20, 2009, which application is incorporated herein by reference.

### BACKGROUND OF THE INVENTION

The first step leading to the initiation of an immune response is the recognition of antigen fragments presented in association with major histocompatibility complex (MHC) molecules. Recognition of antigens can occur directly when the antigens are associated with the MHC on the surface of foreign cells or tissues, or indirectly when the antigens are processed and then associated with the MHC on the surface of professional antigen presenting cells (APC). Resting T lymphocytes that recognize such antigen-MHC complexes become activated via association of these complexes with the T cell receptor (Jenkins et al., J. Exp. Med. 165, 302-319, 1987; Mueller et al., J. Immunol. 144, 3701-3709, 1990). A living organism generally does not display immune response to a self-composing antigen. This is called natural or innate immunological tolerance. On the other hand, even if an antigen is originally heterogeneous to a living organism, it may not react to the immune response which is displayed on dosing of the antigen, depending on when it is dosed, how it is dosed and in what form it is dosed. This is called acquired tolerance. If T cells are only stimulated through the T cell receptor, without receiving an additional costimulatory signal, they become nonresponsive, anergic, or die, resulting in downmodulation of the immune response, and tolerance to the antigen. (Van Gool et al., Eur. J. Immunol. 29(8):2367-75, 1999; Koenen et al., Blood 95(10):3153-61, 2000). However, if the T cells receive a second signal, termed costimulation, T cells are induced to proliferate and become functional (Lenschow et al., Annu. Rev. Immunol 14:233, 1996). The self/non-self recognition is thought to occur at the interaction level of antigen presenting cells (e.g. dendritic cells or macrophages), and T lymphocytes.

Conventional clinical strategies for general long-term immunosuppression in disorders associated with an undesired immune response (e.g., autoimmune disease, graft rejection) are based on the long-term administration of broad acting immunosuppressive drugs, for example, signal 1 blockers such as for example cyclosporin A (CsA), FK506 (tacrolimus) and corticosteroids. Long-term use of high doses of these drugs can also have toxic side-effects. Moreover, even in those patients that are able to tolerate these drugs, the requirement for life-long immunosuppressive drug therapy carries a significant risk of severe side effects, including tumors, serious infections, nephrotoxicity and metabolic disorders (Penn 2000; Fishman et al. 1998).

Methods of inducing antigen-specific tolerance have been developed, including cell coupling of an antigen or peptide. For example, in one method, peptide induced cell coupled tolerance involved collection, separation and treatment of peripheral blood cells with disease specific autoantigens and the ethylene carbodiimide (ECDI) coupling reagent under sterile GMP conditions, and subsequent re-infusion into the donor/patient. This process is costly and must be conducted under closely monitored conditions by skilled practioners and is limited in the number of centers that can conduct the procedure. The use of red blood cells as the carrier cell type expands the potential source to include allogeneic donors thus increasing the supply of source cells dramatically and potentially expanding the delivery of this therapy to any setting certified for blood transfusion. Conventional methods also include the use of ethylene carbodiimide fixed autologous splenocytes. These cells express the peptide for which antigen-specific tolerance is sought. However, collection and preparation of sufficient numbers of cells represents a significant hurdle to the broad utilization of this technology for treating human autoimmune disease, transplant rejection and allergic or hyperimmune responses. These approaches have significant potential limitations in terms of supply of source cells and necessity for tissue type matching to minimize immune response to the carrier. In addition the local treatment of the cells to couple autoantigens via EDCI presents a significant quality control issue.

### SUMMARY OF THE INVENTION

Achievement of immune tolerance is desirable in certain instances, for example, autoimmune disease, transplant rejection and allergic or hyperimmune responses. Accordingly, there is a need for improved approaches that are capable of efficiently inducing long-term immune tolerance without the need for administration of high initial doses of immunosuppressive drugs, or the use of biological material as a carrier.

In one embodiment, the present invention provides a composition for induction of antigen-specific tolerance comprising a carrier particle attached thereto an apoptotic signaling molecule and an antigenic peptide. In another embodiment, the present invention provides a composition for induction of antigen-specific tolerance comprising a carrier particle attached thereto an antigenic peptide. In a preferred embodiment, the carrier particle is a polystyrene particle. In one aspect, the composition induces antigen-specific tolerance in a subject. Where desired, the antigenic peptide can be an autoimmune antigen, a transplantation antigen or an allergen. For instance, the antigenic peptide is myelin basic protein, acetylcholine receptor, endogenous antigen, myelin oligodendrocyte glycoprotein, pancreatic beta-cell antigen, insulin, glutamic acid decarboxylase (GAD), collagen type 11, human carticlage gp39, fp130-RAPS, proteolipid protein, fibrillarin, small nucleolar protein, thyroid stimulating factor receptor, histones, glycoprotein gp70, pyruvate dehydrogenase dehyrolipoamide acetyltransferase (PCD-E2), hair follicle antigen or human tropomyosin isoform 5. In another aspect, the antigenic peptide is coupled to the carrier by a conjugate molecule. In yet another aspect, the apoptotic signaling molecule is a scavenger receptor ligand such as annexin-1, annexin-5, phosphatidyl serine, cholesterol, milk fat globule-EGF-factor 8 (MFG-E8), or Fas-ligand. Where desired, the antigenic peptide can be fused to the apoptotic signalling molecule. In yet another aspect, the carrier comprises a quantum dot. In some instances, the carrier is a dendrimer, liposome or micelle. The carrier can also be a nanoparticle or microparticle is less than 1000 microns in diameter. The nanoparticle or microparticle can be biodegradable.

The present invention also provides a method of reducing an antigen-specific immune response in a subject. The method involves the step of administering to said subject a composition for induction of antigen-specific tolerance. In one embodiment, the composition comprises a carrier particle attached thereto an apoptotic signaling molecule and an antigenic peptide, wherein said composition reduces an antigen-specific immune response in a subject. In another embodiment, the composition comprises a carrier particle attached thereto an antigenic peptide, wherein said composition reduces an antigen-specific immune response in a subject. In a preferred embodiment, the carrier particle is a polystyrene particle. Where desired, the antigenic peptide utilized in the subject method is an autoimmune antigen, a transplantation antigen, or an allergen. In some instances, the autoimmune antigen can be one to which the subject mounts an immune response.

The present invention further provides a method of treating a subject having an autoimmune disorder comprising administering to the subject a composition comprising a nanoparticle or microparticle. The nanoparticle or microparticle comprise (a) an inherent or added apoptotic signaling molecule; and (b) a pathogenic antigen.

The present invention also provides a method for ameliorating a demyelinating disorder in a subject in need of utilizing any of the composition disclosed herein.

Further provided in the present invention is a kit for inducing antigen specific tolerance comprising: (a) a carrier particle; and (b) an antigenic peptide bound to the carrier particle.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the mean clinical score for clinical signs of EAE following treatment using peptides conjugated to an artificial carrier. The graph shows that polystyrene microspheres coupled with the PLP peptide provided protection from PLP₁₃₉₋₁₅₁/CFA induced EAE and abrogated the relapse of active EAE in SJL mice.

FIG. 2 depicts the effect of administration of peptide-coupled polystyrene microspheres either prior to, or after induction of PLP₁₃₉₋₁₅₁ induced EAE in mice. (A) Pre-treatment with peptide-coupled microspheres prior to priming with PLP₁₃₉₋₁₅₁+Complete Freund's Adjuvant (CFA); (B) Pre-treatment with peptide-coupled microspheres prior to priming with PLP₁₇₈₋₁₉₁+Complete Freund's Adjuvant (CFA); (C) Post-treatment with peptide-coupled microspheres following priming with PLP₁₃₉₋₁₅₁+Complete Freund's Adjuvant (CFA).

FIG. 3 depicts the effect of administration of peptide-coupled polystyrene microspheres on delayed type-hypersensitivity ear swelling in a mouse model.

FIG. 4 depicts the effect of administration of peptide-coupled polystyrene microspheres on CNS infiltration of leukocytes into the CNS. Leukocyte markers were assayed from spinal cord slices and stained for: (A) cellularity; (B) CD4+CD3+ cells, and (C) Foxp3+ cells.

FIG. 5 depicts the effect of administration of peptide-coupled polystyrene microspheres on splenectomized mice.

### DETAILED DESCRIPTION OF THE INVENTION

Methods for induction of antigen-specific tolerance are desirable to prevent or diminish immune reactions in several instances, including treatment of autoimmune disease, transplant rejection and allergic or hyperimmune responses. The present invention utilizes a carrier to present antigenic peptides and proteins to the immune system in such a way as to induce antigen-specific tolerance. Antigen presenting cells, such as dendritic cells and macrophages, ordinarily trigger an immune system cascade, but these same cells are capable of inducing tolerance when the antigen is presented in the absence of costimulatory molecules and/or the secretion of inflammatory cytokines (Duperrier, K. et al. “Immunosuppressive agents mediate reduced allostimulatory properties of myeloid-derived dendritic cells despite induction of divergent molecular phenotypes”. Mol Immunol 42 (2005), 1531-40; Piemonti, L. et al. “Glucocorticoids affect human dendritic cell differentiation and maturation”. J Immunol 162 (1999), 6473-81). The carrier of the present invention may be bound to an antigen conjugated to a substance (e.g. ethylene carbodiimide or ECDI) which would allow the particle to be perceived as a self antigen by an antigen presenting cell (APC) of the host reticuloendothelial system or directly by T-cells and allow presentation of the associated antigens in a tolerance-inducing manner. Without being bound by theory, this tolerance may occur by presentation of antigen without associated upregulation of molecules involved in immune cell stimulation (for example, MHC class I/II or costimulatory molecules).

In some embodiments, an inert carrier, such as those described below, are effective to induce antigen-specific tolerance and/or prevent the onset of an immune related disease (such as EAE in a mouse model) and/or diminish the severity of a pre-existing immune related disease. In some embodiments, the compositions and methods of the present invention can cause T cells to undertake early events associated with T-cell activation, but do not allow T-cells to acquire effector function. For example, administration of compositions of the present invention can result in T-cells having a quasi-activated phenotype, such as CD69 and/or CD44 upregulation, but do not display effector function, such as indicated by a lack of IFN-7 or IL-17 synthesis. In some embodiments, administration of compositions of the present invention results in T-cells having a quasi-activated phenotype without having conversion of naive antigen-specific T-cells to a regulatory phenotype, such as those having CD25⁺/Foxp3⁺ phenotypes.

In some instances, the carrier is further conjugated to a molecule that mimics a tolerogenic signal. The addition of an apoptotic signaling molecule is thought to specifically signal to an APC a non-dangerous apoptotic uptake signal which indicates to the host that the associated antigens are self antigens and result in a tolerizing response. In other instances, the carrier particle does not include a separate apoptotic signaling molecule. Without being bound by theory, the carrier particle carrying an antigen specific peptide or protein targets immature B and T cells (e.g., in the spleen, bone marrow or lymph nodes) to affect tolerance.

The invention is useful for treatment of immune related disorders such as autoimmune disease, transplant rejection and allergic reactions. Substitution of a synthetic, biocompatible carrier system capable of carrying a cellular substrate to induce an antigen-specific tolerance response could lead to ease of manufacturing, broad availability of therapeutic agents, increase uniformity between samples, increase the number of potential treatment sites and dramatically reduce the potential for allergic responses to a carrier cell.

As used herein, the term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages. Immune cells involved in the immune response include lymphocytes, such as B cells and T cells (CD4⁺, CD8⁺, Th1and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes.

As used herein, the term “anergy,” “tolerance,” or “antigen-specific tolerance” refers to insensitivity of T cells to T cell receptor-mediated stimulation. Such insensitivity is generally antigen-specific and persists after exposure to the antigenic peptide has ceased. For example, anergy in T cells is characterized by lack of cytokine production, e.g., IL-2. T-cell anergy occurs when T cells are exposed to antigen and receive a first signal (a T cell receptor or CD-3 mediated signal) in the absence of a second signal (a costimulatory signal). Under these conditions, re-exposure of the cells to the same antigen (even if re-exposure occurs in the presence of a costimulatory molecule) results in failure to produce cytokines and subsequently failure to proliferate. Thus, a failure to produce cytokines prevents proliferation. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. 1992 Science. 257:1134).

As used herein, the term “immunological tolerance” refers to methods performed on a proportion of treated subjects in comparison with untreated subjects where: a) a decreased level of a specific immunological response (thought to be mediated at least in part by antigen-specific effector T lymphocytes, B lymphocytes, antibody, or their equivalents); b) a delay in the onset or progression of a specific immunological response; or c) a reduced risk of the onset or progression of a specific immunological response. “Specific” immunological tolerance occurs when immunological tolerance is preferentially invoked against certain antigens in comparison with others.

Various aspects of the invention are described in further detail in the following subsections.

### Carriers

The antigen-specific tolerance inducing compositions of the present invention may be produced with any of a large variety of carriers including, but not limited to, particles, beads, branched polymers, dendrimers, or liposomes. Preferably the carrier is particulate, and generally spherical, ellipsoidal, rod-shaped, globular, or polyhedral in shape. Alternatively, however, the carrier may be of an irregular or branched shape. In preferred embodiments, the carrier is composed of material which is biodegradable. It is further preferred that the carrier have a net neutral or negative charge, in order to reduce non-specific binding to cell surfaces which, in general, bear a net negative charge. The carriers may be capable of being conjugated, either directly or indirectly, to an antigen to which tolerance is desired (also referred to herein as an antigen-specific peptide, antigenic peptide, autoantigen, inducing antigen or tolerizing antigen). In some instances, the carrier will have multiple binding sites in order to have multiple copies of the antigen-specific peptide exposed and increase the likelihood of a tolerance response. The carrier may have one antigenic peptide on the carrier surface or multiple different antigenic peptides on the surface. Alternatively, however, the carrier may have a surface to which conjugating moieties may be adsorbed without chemical bond formation.

In some instances, the antigen-specific peptide is delivered to antigen presenting cells (APCs), such as dendritic cells (DCs) or macrophages, where lymphocytes are undergoing maturation (e.g. spleen, bone marrow, thymus and lymph nodes). There are resident APCs and DCs, for example, in spleen, bone marrow, thymus and lymph nodes. Alternatively, the antigen-specific peptide may be delivered to peripheral APCs or DCs, where they first internalize the carriers and then migrate to sites of lymphocyte maturation (e.g. spleen, bone marrow, thymus or lymph nodes) to activate a tolerance response. This generally occurs within 1-3 days. Resident APCs at sites of lymphocyte maturation may be utilized as targets.

The overall size and weight of the carriers are important considerations. Preferably, the carriers are microscopic or nanoscopic in size, in order to enhance solubility, avoid possible complications caused by aggregation in vivo and to facilitate pinocytosis. Particle size can be a factor for uptake from the interstitial space into areas of lymphocyte maturation.

In various embodiments, the largest cross-sectional diameters of the composition of the invention are less than about 1,000 μm, 500 μm, 100 μm, 50 μm, 25 μm, 20 μm, 15 μm, 10 μm, 5 μm, 1 μm, 500 nm, 400 nm, 300 nm, 200 nm or 100 nm. The composition of the present invention may be chosen to maximize delivery to lymphocytes, for example, immature lymphocyte such as those found in the spleen, thymus, bone marrow or lymph nodes. In some embodiments, carriers have maximum diameters of about 5-80 nm. Alternatively, carriers may have maximum diameters of about 10-70 nm, or 20-60 nm, or 30-50 nm. In some embodiments, the overall weights of the carriers are less than about 10,000 kDa, less than about 5,000 kDa, or less than about 1,000, 500, 400, 300, 200 or 100 kDa.

Preferably the particle surface is composed of a material that minimizes non-specific or unwanted biological interactions. Interactions between the particle surface and the interstitium may be a factor that plays a role in lymphatic uptake. The particle surface may be coated with a material to prevent or decrease non-specific interactions. Steric stabilization by coating particles with hydrophilic layers such as poly(ethylene glycol) (PEG) and its copolymers such as PLURONICS (including copolymers of poly(ethylene glycol)-bl-poly(propylene glycol)-bl-poly(ethylene glycol)) may reduce the non-specific interactions with proteins of the interstitium as demonstrated by improved lymphatic uptake following subcutaneous injections. All of these facts point to the significance of the physical properties of the particles in terms of lymphatic uptake. Biodegradable polymers may be used to make all or some of the polymers and/or particles and/or layers. Biodegradable polymers may undergo degradation, for example, by a result of functional groups reacting with the water in the solution. The term “degradation” as used herein refers to becoming soluble, either by reduction of molecular weight or by conversion of hydrophobic groups to hydrophilic groups. Polymers with ester groups are generally subject to spontaneous hydrolysis, e.g., polylactides and polyglycolides. Many peptide sequences subject to specific enzymatic attack are known, e.g., as degraded by collagenases or metalloproteinases: sequences that are degraded merely by biological free radical mechanisms are not specifically degraded. Polymers with functional groups that are oxidation-sensitive will be chemically altered by mild oxidizing agents, with a test for the same being enhanced solubilization by exposure to 10% hydrogen peroxide for 20 h in vitro.

Carriers of the present invention may also contain additional components. For example, carriers may have imaging agents incorporated or conjugated to the carrier. An example of a carrier nanosphere having an imaging agent that is currently commercially available is the Kodak X-sight nanospheres. Inorganic quantum-confined luminescent nanocrystals, known as quantum dots (QDs), have emerged as ideal donors in FRET applications: their high quantum yield and tunable size-dependent Stokes Shifts permit different sizes to emit from blue to infrared when excited at a single ultraviolet wavelength. (Bruchez, et al., Science, 1998, 281, 2013; Niemeyer, C. M Angew. Chem. hit. Ed. 2003, 42, 5796; Waggoner, A. Methods Enzymol. 1995, 246, 362; Brus, L. E. J. Chem. Phys. 1993, 79, 5566). Quantum dots, such as hybrid organic/inorganic quantum dots based on a class of polymers known as dendrimers, may used in biological labeling, imaging, and optical biosensing systems. (Lemon, et al., J. Am. Chem. Soc. 2000, 122, 12886). Unlike the traditional synthesis of inorganic quantum dots, the synthesis of these hybrid quantum dot nanoparticles does not require high temperatures or highly toxic, unstable reagents. (Etienne, et al., Appl. Phys. Lett. 87, 181913, 2005).

### Microbead or Nanobead Carriers

In some embodiments, the antigen-specific tolerance inducing compositions of the present invention comprise a carrier which is a microparticle or nanoparticle. In some instances, the microparticle or nanoparticle is a substantially spherical bead or a porous bead.

Carrier particles can be formed from a wide range of materials. The particle is preferably composed of a material suitable for biological use. For example, particles may be composed of glass, silica, polyesters of hydroxy carboxylic acids, polyanhydrides of dicarboxylic acids, or copolymers of hydroxy carboxylic acids and dicarboxylic acids. More generally, the carrier particles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, carrier particles can be quantum dots, or composed of quantum dots, such as quantum dot polystyrene particles (Joumaa et al. (2006) Langmuir 22:1810-6). Carrier particles including mixtures of ester and anhydride bonds (e.g., copolymers of glycolic and sebacic acid) may also be employed. For example, carrier particles may comprise materials including polyglycolic acid polymers (PGA), polylactic acid polymers (PLA), polysebacic acid polymers (PSA), poly(lactic-co-glycolic) acid copolymers (PLGA), poly(lactic-co-sebacic) acid copolymers (PLSA), poly(glycolic-co-sebacic) acid copolymers (PGSA), etc. Other biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include PLA, PGA, and PLGA polymers. Biocompatible but non-biodegradable materials may also be used in the carrier particles of the invention. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed.

Suitable beads which are currently available commercially include polystyrene beads such as FluoSpheres (Molecular Probes, Eugene, Oreg.).

In one embodiment, microparticles or nanoparticles are taken up by APC's. The size of the microparticle or nanoparticle is preferably in the range to trigger phagocytosis or pinocytosis in the APC. In some embodiments, the microparticle or nanoparticle is in a range of about 100 nm to 50 μm, 1 μm to 40 μm, 5 μm to 30 μm or 10 μm to 20 μm. In some embodiments, the microparticle or nanoparticle is less than 100 μm, 50 μm, 25 μm, 20 μm, 15 μm, 10 μm, 5 μm, 1 μm, 500 nm or 100 nm. In other embodiments, the microparticle or nanoparticle is more than 10 nm, 50 nm, 100 nm, 500 nm, 600 nm, 700 nm, 800 nm, 900 nm or 1 μm.

In one embodiment, microparticles and nanoparticles are taken up in areas having immature lymphocytes (e.g. spleen, bone marrow, thymus or lymph nodes). As documented herein, size is related to carrier uptake and retention in areas having immature lymphocytes. It is desirable to obtain both efficient uptake and retention, since carrier properties, such as size and surface characteristics, can have conflicting effects. In general, smaller particles have better uptake than larger particles but lower retention. Carriers with a size of about 5 nm to about 10 μm diameter are preferred; artisans will immediately appreciate that all the ranges and values within the explicitly stated ranges are contemplated, e.g., 25 nm, 50 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm or 10 μm. The nanoparticles may be made in a collection that of particles that has a mean diameter from about 5 to about 100 nm; artisans will immediately appreciate that all the ranges and values within the explicitly stated ranges are contemplated, e.g., from about 10 to about 70 nm. The size distribution of such a collection of particles can be controlled so that a coefficient of variation (standard deviation divided by mean particle size) around a mean diameter of a collection of the particles may be less than about 50, about 35, about 20, about 10, or about 5 nm A person of skill in the art will immediately appreciate that all the ranges and values within the explicitly stated ranges are contemplated.

Physical properties are also related to a nanoparticle's usefulness after uptake and retention in areas having immature lymphocytes. These include mechanical properties such as rigidity or rubberiness. Some embodiments are based on a rubbery core, e.g., a polypropylene sulfide) (PPS) core with an overlayer, e.g., a hydrophilic overlayer, as in PEG, as in the PPS-PEG system recently developed and characterized for systemic (but not targeted or immune) delivery. The rubbery core is in contrast to a substantially rigid core as in a polystyrene or metal nanoparticle system. The term rubbery refers to certain resilient materials besides natural or synthetic rubbers, with rubbery being a term familiar to those in the polymer arts. For example, cross-linked PPS can be used to form a hydrophobic rubbery core. PPS is a polymer that degrades under oxidative conditions to polysulfoxide and finally polysulfone, transitioning from a hydrophobic rubber to a hydrophilic, water-soluble polymer. Other sulfide polymers may be adapted for use, with the term sulfide polymer referring to a polymer with a sulfur in the backbone of the mer. Other rubbery polymers that may be used are polyesters with glass transition temperature under hydrated conditions that is less than about 37° C. A hydrophobic core can be advantageously used with a hydrophilic overlayer since the core and overlayer will tend not to mingle, so that the overlayer tends to stericly expand away from the core. A core refers to a particle that has a layer on it. A layer refers to a material covering at least a portion of the core. A layer may be adsorbed or covalently bound. A particle or core may be solid or hollow. Rubbery hydrophobic cores are advantageous over rigid hydrophobic cores, such as crystalline or glassy (as in the case of polystyrene) cores, in that higher loadings of hydrophobic drugs can be carried by the particles with the rubbery hydrophobic cores.

Another physical property is the surface's hydrophilicity. A hydrophilic material may have a solubility in water of at least 1 gram per liter when it is uncrosslinked. Steric stabilization of particles with hydrophilic polymers can improve uptake from the interstitium by reducing non-specific interactions; however, the particles' increased stealth nature can also reduce internalization by phagocytic cells in areas having immature lymphocytes. The challenge of balancing these competing features has been met, however, and this application documents the creation of nanoparticles for effective lymphatic delivery to DCs and other APCs in lymph nodes. Some embodiments include a hydrophilic component, e.g., a layer of hydrophilic material. Examples of suitable hydrophilic materials are one or more of polyalkylene oxides, polyethylene oxides, polysaccharides, polyacrylic acids, and polyethers. The molecular weight of polymers in a layer can be adjusted to provide a useful degree of steric hindrance in vivo, e.g., from about 1,000 to about 100,000 or even more; artisans will immediately appreciate that all the ranges and values within the explicitly stated ranges are contemplated, e.g., between 10,000 and 50,000.

The nanoparticles may incorporate functional groups for further reaction. Functional groups for further reaction include electrophiles or nucleophiles; these are convenient for reacting with other molecules. Examples of nucleophiles are primary amines, thiols, and hydroxyls. Examples of electrophiles are succinimidyl esters, aldehydes, isocyanates, and maleimides.

In preferred embodiments, carrier beads are employed having an average diameter of about 5-1000 nm, 10-400 nm, 20-200 nm, 30-100 nm or about 40-50 nm.

Antigen-specific tolerance may be induced through use of microparticles or nanoparticles as described which are bound to one or more antigenic peptides.

In one series of embodiments, the present invention provides compositions and methods for the induction of tolerance using antigenic peptide attached to a nanoparticle carrier particle. In some instances, the carrier also contains an apoptotic signaling molecule. The carrier particle may be solid, hollow, or porous.

### Polystyrene Beads

Polystyrene beads have been found to elicit an antigen-specific tolerance effect for antigenic peptides bound to the surface without the need for an apoptotic signaling molecule. In one embodiment, the invention provides crosslinked, functionalized polystyrene beads, having excellent properties, such as exceptional uniformity in bead size distribution, pore size, density, swelling properties and/or tolerance to solvents and reagents typically used in oligomer synthesis. In some preferred embodiments, the beads have superior loading characteristics. In some preferred embodiments, the beads have a loading capability of at least about 50 μmole per gram of bead; of at least about 100 μmole per gram of bead; of at least about 150 μmole per gram of bead; of at least about 200 μmole per gram of bead; of at least about 250 μmole per gram of bead; of at least about 300 μmole per gram of bead; of at least about 350 μmole per gram of bead; of at least about 400 μmole per gram of bead; or at least about 450 μmole per gram of bead. In some embodiments, the bead has a loading capability of from about 100 μmole per gram of bead to about 350 μmole per gram of bead.

Polystyrene particles in the range of 40-50 nm are known to trigger a danger signal recognized by dendritic cells (DCs). Polystyrene beads are known to accumulate in lymph nodes at intermediate sizes (40 nm) more than smaller (20 nm) and larger (>100 nm) sizes. In some instances, polystyrene particles between 10-100 nm, 20-80 nm, 30-70 nm, or 40-50 nm may be desired.

The polystyrene bead size may be important in triggering a signal for DCs because DCs have evolved to recognize viral size ranges. The bead size, therefore, would control successful DC targeting, with correctly sized beads being recognized by DCs in the periphery. Additionally, the structure of the spleen lends itself to uptake of the particles by macrophages.

In various embodiments, the largest cross-sectional diameters of the composition of the invention are less than about 1,000 μm, 500 μm, 100 μm, 50 μm, 25 μm, 20 μm, 15 μm, 10 μm, 5 μm, 1 μm, 500 nm, 400 nm, 300 nm, 200 nm or 100 nm. The composition of the present invention may be chosen to maximize delivery to lymphocytes, for example, immature lymphocyte such as those found in the spleen, thymus, bone marrow or lymph nodes. In some embodiments, carriers have maximum diameters of about 10-500 nm. Alternatively, carriers may have maximum diameters of about 100-500 nm, or 250-500 nm, or 300-500 nm. In some embodiments, the overall weights of the carriers are less than about 10,000 kDa, less than about 5,000 kDa, or less than about 1,000, 500, 400, 300, 200 or 100 kDa.

In one series of embodiments, the present invention provides compositions and methods for the induction of tolerance using antigenic peptide attached to a polystyrene bead. In some instances, the antigenic peptide is linked to the polystyrene bead via the N-terminus of the antigenic peptide to the carboxyl sites on the polystyrene beads. In some instances, the carrier also contains an apoptotic signaling molecule.

### Branched Polymer Carriers/Dendrimers

In some embodiments, the tolerance inducing compositions of the present invention comprises a carrier which is a branched polymer, such as a dendrimer. Branched polymers have numerous chain-ends or termini which can be functionalized and, therefore, can be conjugated to a multiplicity of tolerance inducing complexes, either directly or indirectly through conjugating moieties.

Some polymer systems are themselves nanoparticulate and are included in the term nanoparticle. For example, dendrimers are a class of polymer that can be nanoparticulate in the nm range. These polymers comprise a high number of functional groups at their surface, for example which have been used to conjugate to biomolecules and other groups. Analogously, antigen could be conjugated to the dendrimer surface. Moreover, the functional groups on the dendrimer surface could be optimized for complement activation, for example by hydroxylation. Some dendrimer-DNA complexes have been demonstrated to activate complement. Dendrimers represent an interesting nanoparticulate chemistry that could be adapted for lymphatic targeting using the techniques described herein, for antigen conjugation, and for complement activation, e.g., as in U.S. Pat. Pub. Nos. 2004/0086479, 2006/0204443, and in U.S. Pat. Nos. 6,455,071 and 6,998,115, which are hereby incorporated by reference herein to the extent they do not contradict what is explicitly disclosed.

On the other hand, dendrimers have a shape that is highly dependent on the solubility of its component polymers in a given environment, and can change dramatically according to the solvent or solutes around it, e.g., changes in temperature, pH, ion content, or after uptake by a DC. In contrast, nanoparticles that have physical dimensions that are relatively more stable than dendrimers or other merely branched polymer systems can be useful for storage purposes or as related to or biological activity, e.g., a solid core with a hydrophilic corona will consistently present the corona to its environment. Accordingly, some embodiments of nanoparticles rely on particles that are not dendrimers, or that have a core that is a solid and/or have a core that is a cross-linked hydrogel. A PPS-based nanoparticle is not a dendrimer and has a solid core.

Dendrimers, also known as arborols, cascade molecules, dendritic polymers, or fractal polymers, are highly branched macromolecules in which the branches emanate from a central core. Dendrimers can be made from various materials, including, but not limited to, polyamidoamine, polyamidoalcohol, polyalkyleneimine such as polypropyleneimine or polyethyleneimine, polyalkylene such as polystyrene or polyethylene, polyether, polythioether, polyphosphonium, polysiloxane, polyamide, polyaryl polymer, or combinations thereof. Dendrimers have also been prepared from amino acids (e.g., polylysine). Preferably, dendrimers are employed which terminate in carboxyl or other negatively charged reactive groups in order to facilitate conjugation.

Dendrimers are known in the art and are chemically defined globular molecules, generally prepared by stepwise or reiterative reaction of multifunctional monomers to obtain a branched structure (see, e.g., Tomalia et al. (1990) Angew. Chem. Int. Ed. Engl. 29:138-75). A variety of dendrimers are known, e.g., amine-terminated polyamidoamine, polyethyleneimine and polypropyleneimine dendrimers. Exemplary dendrimers useful in the present invention include “dense star” polymers or “starburst” polymers such as those described in U.S. Pat. Nos. 4,587,329; 5,338,532; and 6,177,414, including poly(amidoamine) dendrimers (“PAMAM”). Still other multimeric spacer molecules suitable for use within the present invention include chemically-defined, non-polymeric valency platform molecules such as those disclosed in U.S. Pat. No. 5,552,391; and PCT application publications WO 00/75105, WO 96/40197, WO 97/46251, WO 95/07073, and WO 00/34231. Many other suitable multivalent spacers can be used and will be known to those of skill in the art. For example, dendrimers and their use are described in US Pat App No. 20070238678, which is hereby incorporated by reference in its entirety.

Such dendrimers include but are not limited to polyamidoamine (PAMAM) dendrimers, poly(propyleneimine) (PPI) dendrimers, poly(triazine)dendrimers, poly(ether-hydroxylamine) (PEHAM) dendrimers, which may have their Z groups modified or selected to force the chelating agents exclusively into the dendritic polymer interior or in combination with encapsulation, allow association with the surface of the dendritic polymer. Examples of some such Z surfaces are those which do not interact with the ligand; such Z groups are hydroxyl, ester, acid, ether, carboxylic salts, alkyls, glycols, such as for example hydroxyl groups especially those from amidoethanol, amidoethylethanolamine, tris(hydroxymethyl)amine, carbomethoxypyrrolidinone, amido, thiourea, urea, carboxylate, succinamic acid and polyethylene glycol or primary or primary, secondary or tertiary amine groups with or without hydroxyl alkyl modifications. Other suitable surface groups may include any such functionality that would allow associative attachment (associate with) the dendritic polymer surface and include but are not limited to receptor mediated targeting groups (e.g., folic acid, antibodies, antibody fragments, single chain antibodies, proteins, peptides, oligomers, oligopeptides, or genetic materials) or other functionality that would facilitate biocompatibility, biodistribution, solubility or modulate toxicity. In a preferred embodiment, the dendrimers contain amino and/or carboxy binding sites on the surface.

Suitable dendrimers which are currently available commercially include polyamidoamine dendrimers such as Starburst™ dendrimers (Dendritech, Midland, Mich.). The Starburst™ dendrimers terminate in either amine groups or carboxymethyl groups which may be used, with or without further modification, and with or without interposing conjugating moieties, to conjugate antigen peptides and proteins to the surface of these carriers.

In one method of dendrimer production, dendrimers are synthesized outward from a core molecule by sequential addition of layers of monomers. The first round of dendrimer synthesis adds a single layer or “generation” of monomers to the core, with each monomer having at least one free, reactive terminus. Each subsequent round of polymerization results in the expansion of the dendrimer by one layer and increases the number of free, reactive termini. This process can be repeated numerous times to produce dendrimers of desired diameter or mass. As the density of the branches increases, the outermost branches arrange themselves in the form of a sphere surrounding a lower density core. See, for example, U.S. Pat. No. 5,338,532, which is hereby incorporated by reference in its entirety. In addition, by varying the shape of the core molecules, dendrimers may be produced in rod-shaped, disk-like, and comb-like forms. The resulting dendrimers may possess an arbitrarily large number of free, reactive termini, to which a multiplicity of antigen peptides and proteins may be conjugated, either directly or indirectly. In a preferred embodiment, the dendrimers are spherical or ovoid in shape.

Dendrimers may vary in weight, size, shape and number of terminal reactive groups. For example, dendrimers may range in weight from 100 to 10000 kDa, or 200 to 5000 kDa, or 250 to 2500 kDa. Dendrimers may also range in size from 20 to 1000 nm, 30 to 500 nm, or 50 to 250 nm in the longest dimension.

The use of dendrimers, e.g., PANAM or PPI dendrimers, enables the creation of cationic spherical particles with a specific number of amino binding sites on the surface. The size of these particles can be selected to optimize loading and minimize steric hindrance between surface linked antigen peptides. For example, in most applications PANAM dendrimers of 6-7 generations have been used resulting in particles of 50-125 kDa molecular weights, 60-90 angstrom diameter (roughly similar in size as hemoglobin, IgG or histones), and 100-1500 active surface groups.

Multivalent spacers with a variety of valencies are useful in the practice of the invention, and in various embodiments the multivalent spacer is bound to between about 3 and about 400 nucleic acid moieties, often from 3 to 100, sometimes from 3-50, frequently from 3-10, and sometimes more than 400 nucleic acid moieties. In various embodiments, the multivalent spacer is conjugated to more than 10, more than 25, more than 50, or more than 500 nucleic acid moieties (which may be the same or different). It will be appreciated that, in certain embodiments comprising a multivalent spacer, the invention provides a population with slightly different molecular structures. For example, dendrimers of the present invention may be composed of a somewhat heterogeneous mixture of molecules produced, i.e., comprising different numbers (within or predominantly within a determinable range) of nucleic acid moieties joined to each dendrimer molecule. In a preferred embodiment, the dendrimers are of a similar size and shape, i.e., composed of numbers of nucleic acid moieties that vary within 20%, 15%, 10%, 5%, 2% or 1% of each other.

Non-dendrimer branched polymers may also be employed in the invention, and may be produced from the same general classes of materials as dendrimers. The synthesis of such branched polymers is also well known in the art.

Branched polymers may include at least 5 termini, at least 10 termini, or at least 100 termini. Branched polymers may include between 5 and 500 termini, preferably between 10 and 400 termini and more preferably between 50 and 250 termini.

In some embodiments, the tolerance inducing compositions of the present invention provides for the production of conjugates wherein a tolerance inducing complex is conjugated to a branched or linear polymer.

### Liposome Carriers

In some embodiments, the multimeric antigen peptide or protein conjugate comprises a carrier which is a liposome or micelle. Liposomes, also called lipid vesicles, are aqueous compartments enclosed by lipid membranes, and are typically formed by suspending a suitable lipid in an aqueous medium, and shaking, extruding, or sonicating the mixture to yield a dispersion of vesicles. Various forms of liposomes, including unilamellar vesicles and multilamellar vesicles, may be used in the present invention.

Micellar systems may also display the same useful characteristics as described above, including micelles formed from AB and ABA block copolymers of poly(ethylene glycol) and PPS. When such copolymers are formed with a molecular fraction of poly(ethylene glycol) that is relatively high, e.g., in excess of approx. 40%, then spherical micelles can be expected to form under certain conditions. These micelles can be small, e.g., meeting the size mentioned above for lymphatic entry, and may optionally be grafted with an overlayer of PEG, or otherwise incorporate PEG or other polymers to achieve similar properties. Moreover, they can be conjugated with antigen, as taught herein, danger signals or both at the micelle surface. The block copolymer can terminate in a hydroxyl group, for complement activation, and it is particularly beneficial to have the hydrophilic block terminate in a hydroxyl group, so that this hydroxyl group will be more readily available on the micellar nanoparticle surface for complement binding. Such hydroxylated such surfaces can be tailored to effectively activate complement. A particularly useful hydrophilic block is PEG, terminated in a hydroxyl group. In addition to micelle-forming polymer architectures, block sizes and block size ratios can be selected to form vesicular structures. There also exists a number of other possible chemical compositions of micellar formulations that may be used.

In another series of embodiments, the present invention provides for the production of multimeric antigen peptide and protein conjugates in which a multiplicity of antigen peptides and proteins are conjugated to the outer surface of a liposome.

Liposomes may be prepared from a variety of lipid materials including, but not limited to, lipids of phosphatidyl choline, phosphatidyl serine, phosphatidyl inositol, phosphatidyl glycerol, phosphatidyl ethanolamine, phosphatidic acid, dicetyl phosphate, monosialoganglioside, polyethylene glycol, stearyl armine, ovolecithin and cholesterol, as well as mixtures of these in varying stoichiometries. Liposomes, as used herein, may also be formed from non-lipid amphipathic molecules, such as block copolymers of poly(oxyethylene-b-isoprene-b-oxye-thylene) and the like. In preferred embodiments, the liposomes are prepared from lipids that will form negatively charged liposomes, such as those produced from phosphatidyl serine, dicetyl phosphate, and dimyristoyl phosphatidic acid.

The surfaces of liposomes may also be modified to reduce immunogenicity or to provide convenient reactive groups for conjugation. For example, sialic acid or other carbohydrates, or polyethylene glycol or other alkyl or alkenyl polymers, may be attached to the surface of a liposome to reduce immunogenicity. Alternatively, liposomes may be produced bearing a conjugating moiety such as biotin by inclusion of a small molar percentage of, for example, biotin-X-dipalmitoylphosphatidyle-thanolamine (Molecular Probes, Eugene, Oreg.) in the liposome.

### Means of Conjugating Antigen Peptides and Proteins to a Carrier

A great variety of means, well known in the art, may be used to conjugate antigenic peptides and proteins to carriers. These methods include any standard chemistries which do not destroy or severely limit the biological activity of the antigen peptides and proteins, and which allow for a sufficient number of antigen peptides and proteins to be conjugated to the carrier in an orientation which allows for interaction of the antigen peptide or protein with a cognate T cell receptor. Generally, methods are preferred which conjugate the C-terminal regions of an antigen peptide or protein, or the C-terminal regions of an antigen peptide or protein fusion protein, to the carrier. The exact chemistries will, of course, depend upon the nature of the carrier material, the presence or absence of C-terminal fusions to the antigen peptide or protein, and/or the presence or absence of conjugating moieties.

Functional groups can be located on the particle as needed for availability. One location can be as side groups or termini on the core polymer or polymers that are layers on a core or polymers otherwise tethered to the particle. For instance, examples are included herein that describe PEG stabilizing the nanoparticles that can be readily functionalized for specific cell targeting or protein and peptide drug delivery.

Conjugates such as ethylene carbodiimide (ECDI), hexamethylene diisocyanate, propyleneglycol di-glycidylether which contain 2 epoxy residues, and epichlorohydrin may be used for fixation of peptides or proteins to the carrier surface. Without being bound by theory, ECDI is suspected of carrying out two major functions for induction of tolerance: (a) it chemically couples the protein/peptides to the cell surface via catalysis of peptide bond formation between free amino and free carboxyl groups; and (b) it induces the carrier to mimic apoptotic cell death such that they are picked up by host antigen presenting cells in the spleen and induce tolerance. It is this presentation to host T-cells in a non-immunogenic fashion that leads to direct induction of anergy in autoreactive cells. In addition, ECDI serves as a potent stimulus for the induction of specific regulatory T cells.

In one series of embodiments, the antigen peptides and proteins are bound to the carrier via a covalent chemical bond. For example, a reactive group or moiety near the C-terminus of the antigen (e.g., the C-terminal carboxyl group, or a hydroxyl, thiol, or amine group from an amino acid side chain) may be conjugated directly to a reactive group or moiety on the surface of the carrier (e.g., a hydroxyl or carboxyl group of a PLA or PGA polymer, a terminal amine or carboxyl group of a dendrimer, or a hydroxyl, carboxyl or phosphate group of a phospholipid) by direct chemical reaction. Alternatively, there may be a conjugating moiety which covalently conjugates to both the antigen peptides and proteins and the carrier, thereby linking them together.

Reactive carboxyl groups on the surface of a carrier may be joined to free amines (e.g., from Lys residues) on the antigen peptide or protein, by reacting them with, for example, 1-ethyl-3-[3,9-dimethyl aminopropyl]carbodiimide hydrochloride (EDC) or N-hydroxysuccinimide ester (NHS). Similarly, the same chemistry may be used to conjugate free amines on the surface of a carrier with free carboxyls (e.g., from the C-terminus, or from Asp or Glu residues) on the antigen peptide or protein. Alternatively, free amine groups on the surface of a carrier may be covalently bound to antigen peptides and proteins, or antigen peptide or protein fusion proteins, using sulfo-SIAB chemistry, essentially as described by Arano et al. (1991) Bioconjug. Chem. 2:71-6.

In another embodiment, a non-covalent bond between a ligand bound to the antigen peptide or protein and an anti-ligand attached to the carrier may conjugate the antigen to the carrier. For example, a biotin ligase recognition sequence tag may be joined to the C-terminus of an antigen peptide or protein, and this tag may be biotinylated by biotin ligase. The biotin may then serve as a ligand to non-covalently conjugate the antigen peptide or protein to avidin or streptavidin which is adsorbed or otherwise bound to the surface of the carrier as an anti-ligand. Alternatively, if the antigen peptides and proteins are fused to an immunoglobulin domain bearing an Fc region, as described above, the Fc domain may act as a ligand and protein A, either covalently or non-covalently bound to the surface of the carrier, may serve as the anti-ligand to non-covalently conjugate the antigen peptide or protein to the carrier. Other means are well known in the art which may be employed to non-covalently conjugate antigen peptides and proteins to carriers, including metal ion chelation techniques (e.g., using a poly-His tag at the C-terminus of the antigen peptide or protein or antigen peptide or protein fusion proteins, and a Ni⁺-coated carrier), and these methods may be substituted for those described here.

Conjugation of a nucleic acid moiety to a platform molecule can be effected in any number of ways, typically involving one or more crosslinking agents and functional groups on the nucleic acid moiety and platform molecule. Linking groups are added to platforms using standard synthetic chemistry techniques. Linking groups can be added to nucleic acid moieties using standard synthetic techniques.

### Apoptosis Signaling Molecules

In some embodiments, the tolerance inducing compositions of the present invention contain an apoptosis signaling molecule. The apoptotic signaling molecules serve allow a carrier to be perceived as an apoptotic body by antigen presenting cells of the host, such as cells of the host reticuloendothelial system. This allows presentation of the associated peptide epitopes in a tolerance-inducing manner. Without being bound by theory, this is presumed to prevent the upregulation of molecules involved in immune cell stimulation, such as MHC class I/II, and costimulatory molecules. These apoptosis signaling molecules may also serve as phagocytic markers. For example, apoptosis signaling molecules suitable for the present invention have been described in US Pat App No. 20050113297, which is hereby incorporated by reference in its entirety. Molecules suitable for the present invention include molecules that target phagocytes, which include macrophages, dendritic cells, monocytes and neutrophils.

Molecules suitable as apoptotic signaling molecules act to enhance tolerance of the associated peptides. Additionally, a carrier bound to an apoptotic signaling molecule can be bound by Clq in apoptotic cell recognition (Paidassi et al., (2008) J. Immunol. 180:2329-2338). For example, molecules that may be useful as apoptotic signaling molecules include phosphatidyl serine, annexin-1, annexin-5, milk fat globule-EGF-factor 8 (MFG-E8), or the family of thrombospondins.

Thrombospondins are a family of extracellular proteins that participate in cell-to-cell and cell-to-matrix communication. They regulate cellular phenotype during tissue genesis and repair. In addition, thrombospondin-1 (TSP-1) is expressed on apoptotic cells and is involved in their recognition by macrophages. Thrombospondin-1 is therefore another phagocytic marker that can be used to enhance phagocytosis in accordance with the invention. Macrophages recognize TSP-1 on apoptotic cells via the CD36 molecule, which is present on the surface of macrophages and may also be present on apoptotic cells. While not wishing to be bound by any theory, it is possible that CD36/TSP1 complex on the surface of an apoptotic cell may form a ligand bridging the cell to a complex consisting of alpha(v)beta 3/CD36/TSP1 on macrophages. It is possible that binding of TSP-1 to CD36 is mediated by interaction of the TSR-1 domain of TSP-1 with a conserved domain called CLESH-1 in CD36. In certain embodiments of the invention phagocytosis is enhanced by increasing the level or density of TSP-1, CD36, or a TSP-1/CD36 complex on the surface of a cell or molecule, e.g., by delivering the TSP-1, CD36, or TSP-1/CD36 complex to the cell. In certain embodiments of the invention a TSP-1/CLESH domain complex is delivered to the cell.

Alternatively or additionally, the phagocytic marker may comprise a molecule (e.g., MFG-E8, b2-glycoprotein, etc.) that serves as a bridging agent between macrophages and their targets, or a portion of such a molecule. Such markers may, for example, facilitate recognition of phosphatidyl serine by macrophages or be independently recognized. Other markers that are also known to enhance phagocytosis include protein S, the growth arrest specific gene product GAS-6, and various complement components including, but not limited to, factor B, Clq, and C3. As mentioned above, MFG-E8 is a secreted glycoprotein, which is produced by stimulated macrophages and binds specifically to apoptotic cells by recognizing aminophospholipids such as phosphatidylserine (PS). MFG-E8, when engaged by phospholipids, binds to cells via its RGD (arginine-glycine-aspartate) motif and binds particularly strongly to cells expressing alpha(v)beta(3) integrin, such as macrophages. At least two splice variants of MFG-E8 are known, of which the L variant is believed to be active for stimulating phagocytosis. In certain embodiments of the invention the phagocytic marker comprises the L splice variant of MFG-E8 (MFG-E8-L). In certain embodiments of the invention the phagocytic marker comprises an N-terminal domain of MFG-E8.

Annexin I is another phagocytic marker that may be used according to the present invention. Briefly, the 37 kDa protein annexin I (Anx-1; lipocortin 1) is a glucocorticoid-regulated protein that has been implicated in the regulation of phagocytosis, cell signaling and proliferation, and is postulated to be a mediator of glucocorticoid action in inflammation and in the control of anterior pituitary hormone release. Annexin I expression is elevated in apoptotic cells and appears to play a role in bridging phosphatidylserine on apoptotic cells to phagocytes and to enhancing recognition of apoptotic cells by phagocytes such as macrophages. While not wishing to be bound by any theory, it is possible that the phosphatidylserine receptor on macrophages recognizes either annexin I or a complex containing annexin I and PS, or that annexin I facilitates recognition by aggregating PS into clusters. Additionally, other DC targeting studies use conjugated targeting ligands such as anti-Dec-205 and anti-CD11c to increase DC specificity.

In some embodiments, the apoptotic signaling molecule may be conjugated to the antigen-specific peptide. In some instances, the apoptotic signaling molecule and antigen-specific peptide are conjugated by the creation of a fusion protein. As used herein, a “fusion protein” refers to a protein formed by the fusion of at least one antigen-specific peptide (or a fragment or a variant thereof) to at least one molecule of an apoptotic signaling molecule (or a fragment or a variant thereof). For the creation of fusion proteins, the terms “fusion protein,” “fusion peptide,” “fusion polypeptide,” and “chimeric peptide” are used interchangably. Suitable fragments of the antigen-specific peptide include any fragment of the full-length peptide that retains the function of generating the desired antigen-specific tolerance function of the present invention. Suitable fragments of the apoptotic signaling molecules include any fragment of the full-length peptide that retains the function of generating an apoptotic signal. The present application is also directed to proteins containing polypeptides at least 80%, 85%, 90%, 95%, 96%, 97%, 98% or 99% identical to the reference polypeptide sequence (e.g., the antigen-specific peptide or apoptotic signaling molecule or the fusion protein thereof) set forth herein, or fragments thereof. Variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide. As used herein, “variant”, refers to an antigen-specific peptide, apoptotic signaling molecule or fusion protein thereof differing in sequence from an antigen-specific peptide, apoptotic signaling molecule or fusion protein thereof of the invention, respectively, but retaining at least one functional and/or therapeutic property thereof (e.g., trigger tolerance in an immune system or produce an apoptotic signal). The present invention is also directed to proteins which comprise, or alternatively consist of, an amino acid sequence which is at least 80%, 85%, 90%, 95%, 96%, 97%, 98%, 99% or 100%, identical to, for example, the amino acid sequence of an antigen-specific peptide, apoptotic signaling molecule or fusion protein thereof of the invention.

The fusion protein may be created by various means. One means is by genetic fusion (i.e. the fusion protein is generated by translation of a nucleic acid sequence in which a polynucleotide encoding all or a portion or a variant of an antigen-specific peptide in joined in frame to a polynucleotide encoding all or a portion or a variant of an apoptotic signaling molecule. The two proteins may be fused either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order. This term also refers to conservatively modified variants, polymorphic variants, alleles, mutants, subsequences, and interspecies homologs of the antigens that make up the fusion protein. The fusion protein may also be created by chemical conjugation. Protocols for generation of fusion polypeptides are well known in the art, and include various recombinant means and DNA synthesizers. Alternatively, the apoptotic signaling molecule and antigen-specific peptide fusion protein can also be easily created using PCR amplification and anchor primers that give rise to complementary overhangs between two consecutive gene fragments that can subsequently be annealed and reamplified to generate a chimeric gene sequence. For example, an apoptotic signaling molecule can be fused in-frame with an antigen-specific peptide. In the present invention, either the apoptotic signaling molecule or antigen-specific peptide may be the N-terminal portion of the fusion protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et. al., Gene 40:39-46 (1985); Murphy et al., Proc. Natl. Acad. Sci. USA 83:8258-8262 (1986); U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

### Antigenic Peptides and Proteins

The practitioner has a number of choices for antigens used in the combinations of this invention. The inducing antigen present in the combination contributes to the specificity of the tolerogenic response that is induced. It may or may not be the same as the target antigen, which is the antigen present or to be placed in the subject being treated which is a target for the unwanted immunological response, and for which tolerance is desired.

An inducing antigen of this invention may be a polypeptide, polynucleotide, carbohydrate, glycolipid, or other molecule isolated from a biological source, or it may be a chemically synthesized small molecule, polymer, or derivative of a biological material, providing it has the ability to induce tolerance according to this description when combined with the mucosal binding component.

In certain embodiments of this invention, the inducing antigen is a single isolated or recombinantly produced molecule. For treating conditions where the target antigen is disseminated to various locations in the host, it is generally necessary that the inducing antigen be identical to or immunologically related to the target antigen. Examples of such antigens are most polynucleotide antigens, and some carbohydrate antigens (such as blood group antigens).

Where the target antigen is preferentially expressed on a particular organ, cell, or tissue type, the practitioner again has the option of using an inducing antigen which is identical with or immunologically related to the target antigen. However, there is also the additional option of using an antigen which is a bystander for the target. This is an antigen which may not be immunologically related to the target antigen, but is preferentially expressed in a tissue where the target antigen is expressed. A working theory as to the effectiveness of bystander suppression is that suppression is an active cell-mediated process that down-regulates the effector arm of the immune response at the target cells. The suppressor cells are specifically stimulated by the inducer antigen at the mucosal surface, and home to a tissue site where the bystander antigen is preferentially expressed. Through an interactive or cytokine-mediated mechanism, the localized suppressor cells then down-regulate effector cells (or inducers of effector cells) in the neighborhood, regardless of what they are reactive against. If the effector cells are specific for a target different from the inducing antigen, then the result is a bystander effect. For further elaboration of the bystander reaction and a list of tolerogenic peptides having this effect, the reader is referred to International Patent Publication WO 93/16724. An implication of bystander theory is that one of ordinary skill need not identify or isolate a particular target antigen against which tolerance is desired in order to practice the present invention. The practitioner need only be able to obtain at least one molecule preferentially expressed at the target site for use as an inducing antigen.

In certain embodiments of this invention, the inducing antigen is not in the same form as expressed in the individual being treated, but is a fragment or derivative thereof. Inducing antigens of this invention include peptides based on a molecule of the appropriate specificity but adapted by fragmentation, residue substitution, labeling, conjugation, and/or fusion with peptides having other functional properties. The adaptation may be performed for any desirable purposes, including but not limited to the elimination of any undesirable property, such as toxicity or immunogenicity; or to enhance any desirable property, such as mucosal binding, mucosal penetration, or stimulation of the tolerogenic arm of the immune response. Terms such as insulin peptide, collagen peptide, and myelin basic protein peptide, as used herein, refer not only to the intact subunit, but also to allotypic and synthetic variants, fragments, fusion peptides, conjugates, and other derivatives that contain a region that is homologous (preferably 70% identical, more preferably 80% identical and even more preferably 90% identical at the amino acid level) to at least 10 and preferably 20 consecutive amino acids of the respective molecule for which it is an analog, wherein the homologous region of the derivative shares with the respective parent molecule an ability to induce tolerance to the target antigen.

It is recognized that tolerogenic regions of an inducing antigen are often different from immunodominant epitopes for the stimulation of an antibody response. Tolerogenic regions are generally regions that can be presented in particular cellular interactions involving T cells. Tolerogenic regions may be present and capable of inducing tolerance upon presentation of the intact antigen. Some antigens contain cryptic tolerogenic regions, in that the processing and presentation of the native antigen does not normally trigger tolerance. An elaboration of cryptic antigens and their identification is found in International Patent Publication WO 94/27634.

In certain embodiments of this invention, two, three, or a higher plurality of inducing antigens is used. It may be desirable to implement these embodiments when there are a plurality of target antigens, or to provide a plurality of bystanders for the target. For example, both insulin and glucagon can be mixed with a mucosal binding component in the treatment of diabetes. It may also be desirable to provide a cocktail of antigens to cover several possible alternative targets. For example, a cocktail of histocompatibility antigen fragments could be used to tolerize a subject in anticipation of future transplantation with an allograft of unknown phenotype. Allovariant regions of human leukocyte antigens are known in the art: e.g., Immunogenetics 29:231, 1989. In another example, a mixture of allergens may serve as inducing antigen for the treatment of atopy.

Inducing antigens can be prepared by a number of techniques known in the art, depending on the nature of the molecule. Polynucleotide, polypeptide, and carbohydrate antigens can be isolated from cells of the species to be treated in which they are enriched. Short peptides are conveniently prepared by amino acid synthesis. Longer proteins of known sequence can be prepared by synthesizing an encoding sequence or PCR-amplifying an encoding sequence from a natural source or vector, and then expressing the encoding sequence in a suitable bacterial or eukaryotic host cell.

In certain embodiments of this invention, the combination comprises a complex mixture of antigens obtained from a cell or tissue, one or more of which plays the role of inducing antigen. The antigens may be in the form of whole cells, either intact or treated with a fixative such as formaldehyde, glutaraldehyde, or alcohol. The antigens may be in the form of a cell lysate, created by detergent solubilization or mechanical rupture of cells or tissue, followed by clarification. The antigens may also be obtained by subcellular fractionation, particularly an enrichment of plasma membrane by techniques such as differential centrifugation, optionally followed by detergent solubilization and dialysis. Other separation techniques are also suitable, such as affinity or ion exchange chromatography of solubilized membrane proteins.

In one embodiment, the antigenic peptide or protein is an autoantigen, an alloantigen or a transplantation antigen. In yet another particular embodiment, the autoantigen is selected from the group consisting of myelin basic protein, collagen or fragments thereof, DNA, nuclear and nucleolar proteins, mitochondrial proteins and pancreatic β-cell proteins.

The invention provides for the induction of tolerance to an autoantigen for the treatment of autoimmune diseases by administering the antigen for which tolerance is desired. For example, autoantibodies directed against the myelin basic protein (MBP) are observed in patients with multiple sclerosis, and, accordingly, MBP antigenic peptides or proteins may be used in the invention to be delivered using the compositions of the present invention to treat and prevent multiple sclerosis.

By way of another non-limiting example, an individual who is a candidate for a transplant from a non-identical twin may suffer from rejection of the engrafted cells, tissues or organs, as the engrafted antigens are foreign to the recipient. Prior tolerance of the recipient individual to the intended graft abrogates or reduces later rejection. Reduction or elimination of chronic anti-rejection therapies may be achieved by the practice of the present invention. In another example, many autoimmune diseases are characterized by a cellular immune response to an endogenous or self antigen. Tolerance of the immune system to the endogenous antigen is desirable to control the disease.

In a further example, sensitization of an individual to an industrial pollutant or chemical, such as may be encountered on-the-job, presents a hazard of an immune response. Prior tolerance of the individual's immune system to the chemical, in particular in the form of the chemical reacted with the individual's endogenous proteins, may be desirable to prevent the later occupational development of an immune response.

Allergens are other antigens for which tolerance of the immune response thereto is also desirable.

Notably, even in diseases where the pathogenic autoantigen is unknown, bystander suppression may be induced using antigens present in the anatomical vicinity. For example, autoantibodies to collagen are observed in rheumatoid arthritis and, accordingly, a collagen-encoding gene may be utilized as the antigen-expressing gene module in order to treat rheumatoid arthritis (see e.g. Choy (2000) Curr Opin Investig Drugs 1: 58-62). Furthermore, tolerance to beta cell autoantigens may be utilized to prevent development of type 1 diabetes (see e.g. Bach and Chatenoud (2001) Ann Rev Immunol 19: 131-161).

As another example, auto-antibodies directed against myelin oligodendrocyte glycoprotein (MOG) is observed in autoimmune encephalomyelitis and in many other CNS diseases as well as multiple sclerosis (see e.g. Iglesias et al. (2001) Glia 36: 22-34). Accordingly, use of MOG antigen expressing constructs in the invention allows for treatment of multiple sclerosis as well as related autoimmune disorders of the central nervous system.

Still other examples of candidate autoantigens for use in treating autoimmune disease include: pancreatic beta-cell antigens, insulin and GAD to treat insulin-dependent diabetes mellitus; collagen type 11, human cartilage gp 39 (HCgp39) and gp130-RAPS for use in treating rheumatoid arthritis; myelin basic protein (MBP), proteolipid protein (PLP) and myelin oligodendrocyte glycoprotein (MOG, see above) to treat multiple sclerosis; fibrillarin, and small nucleolar protein (snoRNP) to treat scleroderma; thyroid stimulating factor receptor (TSH-R) for use in treating Graves' disease; nuclear antigens, histones, glycoprotein gp70 and ribosomal proteins for use in treating systemic lupus erythematosus; pyruvate dehydrogenase dehydrolipoamide acetyltransferase (PCD-E2) for use in treating primary billiary cirrhosis; hair follicle antigens for use in treating alopecia areata; and human tropomyosin isoform 5 (hTM5) for use in treating ulcerative colitis.

### Evaluating Tolerance

Combinations can be tested for their ability to promote tolerance by conducting experiments with isolated cells or in animal models.

A proxy for tolerogenic activity is the ability of an intact antigen or fragment to stimulate the production of an appropriate cytokine at the target site. The immunoregulatory cytokine released by T suppressor cells at the target site is thought to be TGF-β (Miller et al., Proc. Natl. Acad. Sci. USA 89:421, 1992). Other factors that may be produced during tolerance are the cytokines IL4 and IL-10, and the mediator PGE. In contrast, lymphocytes in tissues undergoing active immune destruction secrete cytokines such as IL-1, IL-2, IL-6, and γ-IFN. Hence, the efficacy of a candidate inducing antigen can be evaluated by measuring its ability to stimulate the appropriate type of cytokines.

With this in mind, a rapid screening test for tolerogenic epitopes of the inducing antigen, effective mucosal binding components, effective combinations, or effective modes and schedules of mucosal administration can be conducted using syngeneic animals as donors for in vitro cell assays. Animals are treated at a mucosal surface with the test composition, and at some time are challenged with parenteral administration of the target antigen in complete Freund's adjuvant. Spleen cells are isolated, and cultured in vitro in the presence of the target antigen at a concentration of about 50 μg/mL. Target antigen can be substituted with candidate proteins or sub-fragments to map the location of tolerogenic epitopes. Cytokine secretion into the medium can be quantitated by standard immunoassay.

The ability of the cells to suppress the activity of other cells can be determined using cells isolated from an animal immunized with the target antigen, or by creating a cell line responsive to the target antigen (Ben-Nun et al., Eur. J. Immunol. 11:195, 1981). In one variation of this experiment, the suppressor cell population is mildly irradiated (about 1000 to 1250 rads) to prevent proliferation, the suppressors are co-cultured with the responder cells, and then tritiated thymidine incorporation (or MTT) is used to quantitate the proliferative activity of the responders. In another variation, the suppressor cell population and the responder cell population are cultured in the upper and lower levels of a dual chamber transwell culture system (Costar, Cambridge Mass.), which permits the populations to coincubate within 1 mm of each other, separated by a polycarbonate membrane (WO 93/16724). In this approach, irradiation of the suppressor cell population is unnecessary, since the proliferative activity of the responders can be measured separately.

In embodiments of the invention where the target antigen is already present in the individual, there is no need to isolate the antigen or precombine it with the mucosal binding component. For example, the antigen may be expressed in the individual in a certain fashion as a result of a pathological condition (such as inflammatory bowel disease or Celiac disease) or through digestion of a food allergen. Testing is performed by giving the mucosal binding component in one or more doses or formulations, and determining its ability to promote tolerization against the antigen in situ.

The effectiveness of compositions and modes of administration for treatment of specific disease can also be elaborated in a corresponding animal disease model. The ability of the treatment to diminish or delay the symptomatology of the disease is monitored at the level of circulating biochemical and immunological hallmarks of the disease, immunohistology of the affected tissue, and gross clinical features as appropriate for the model being employed. Non-limiting examples of animal models that can be used for testing are included in the following section.

The invention contemplates modulation of tolerance by modulating TH1 response, TH2 response, TH17 response, or a combination of these responses. Modulating TH1 response encompasses changing expression of, e.g., interferon-gamma. Modulating TH2 response encompasses changing expression of, e.g., any combination of IL-4, IL-5, IL-10, and IL-13. Typically an increase (decrease) in TH2 response will comprise an increase (decrease) in expression of at least one of IL-4, IL-5, IL-10, or IL-13; more typically an increase (decrease) in TH2 response will comprise an increase in expression of at least two of IL-4, IL-5, IL-10, or IL-13, most typically an increase (decrease) in TH2 response will comprise an increase in at least three of IL-4, IL-5, IL-10, or IL-13, while ideally an increase (decrease) in TH2 response will comprise an increase (decrease) in expression of all of IL-4, IL-5, IL-10, and IL-13. Modulating TH17 encompasses changing expression of, e.g., TGF-beta, IL-6, IL-21 and IL23, and effects levels of IL-17, IL-21 and IL-22.

In the study of the present invention, despite accelerated disease onset, overall disease incidence and severity was reduced over time in CD200KO mice, where reduction in disease symptoms correlated with elevated numbers of regulatory T cells and the presence of high IL-10 secreting splenic myeloid cells later in the disease process. The CD200KO enhanced tolerance to retinal antigen. This result of the CD200KO may be related to the altered phenotype of APC in the respiratory tract compared to wild type and an enhanced Th2 switch in tolerised CD200KO mice. Tolerance induction in the CD200KO mouse was efficient, with up 50% of eyes still protected from disease 28 days post-immunisation (see, e.g., Murphy and Reiner (2002) Nat. Rev. Immunol. 2:933-944; Suri-Payer, et al. (1998) J. Immunol. 160:1212-1218; Thornton and Shevach (2000) J. Immunol 164:183-190; Roncarolo, et al. (2001) Immunol. Rev. 182:68-79; Peiser and Gordon (2001) Microbes Infect. 3:149-159; Gordon (2003) Nat. Rev. Immunol. 3:23-35).

In the studies of the present invention, there was a clear increase in CD11b⁻IL10^{high} cells in the spleens of both sham tolerised and tolerised CD200KO mice at day 28. These cells were distinct from larger populations of CD11b⁺IL10^{low} present in all experimental groups from day 21. The high level of IL-10 detected was endogenous as cells were analysed directly ex vivo without any additional activating stimulus or artificial sequestering of cytokine by brefeldin A or other Golgi inhibitors. Further analysis of these cells indicated that they were CD11c⁻/low, CD45RB^{intermediate} and B220⁻ and had plasmacytoid DC morphology. Tolerogenic plasmacytoid DC with similar phenotype but CD45RB^{high} can be generated by in vitro culture with IL-10, can be isolated from the spleens of normal C57B1/6 mice and are elevated in IL10 transgenic mice. The cells take 3 weeks to differentiate in vitro, and in the studies of the present invention appear in CD200KO spleens 3-4 weeks after disease onset suggesting that prolonged stimulation and/or several rounds of cell division are involved. Bone marrow derived plasmacytoid cells were also tolerogenic and capable of generating antigen specific IL-10 secreting Tregs, in vivo. Significant numbers of CD3⁺CD4⁺IL-10⁺cells were not found in this study, but a trend towards increased numbers of CD3⁺CD4⁺CD25⁺in CD200KO mice was found and this was significant in tolerised groups at all time points. Tregs can have an immunosuppressive effect, e.g., by inhibiting expression of IL-2 or IL-10. Induction of IL-10 and suppression of IL-2 in all groups at day 28 of the study of the present invention is consistent with induction of regulatory T cells during the disease process, and findings linking nasal administration of antigen with induction of Tr1 (see, e.g., Shevach (2002) Nat. Rev. Immunol 2:389400; McGuirk and Mills (2002) Trends Immunol. 23:450455; Herrath and Harrison (2003) Nat. Rev. Immunol. 3:223-232; Bluestone and Abbas (2003) Nat. Rev. Immunol. 3:253-257; Thornton and Shevach (1998) J. Exp. Med. 188:287-296; Jonuleit, et al. (2000) J. Exp. Med. 192:1213-1222; Wakkach, et al. (2003) Immunity 18:605-617).

Tolerance to autoantigens and autoimmune disease is achieved by a variety of mechanisms including negative selection of self-reactive T cells in the thymus and mechanisms of peripheral tolerance for those autoreactive T cells that escape thymic deletion and are found in the periphery. Examples of mechanisms that provide peripheral T cell tolerance include “ignorance” of self antigens, anergy or unresponsiveness to autoantigen, cytokine immune deviation, and activation-induced cell death of self-reactive T cells. In addition, regulatory T cells have been shown to be involved in mediating peripheral tolerance. See, for example, Walker et al. (2002) Nat. Rev. Immunol. 2:11-19; Shevach et al. (2001) Immunol. Rev. 182:58-67. In some situations, peripheral tolerance to an autoantigen is lost (or broken) and an autoimmune response ensues. For example, in an animal model for EAE, activation of antigen presenting cells (APCs) through TLR innate immune receptors was shown to break self-tolerance and result in the induction of EAE (Waldner et al. (2004) J. Clin. Invest. 113:990-997).

Accordingly, in some embodiments, the invention provides methods for increasing antigen presentation while suppressing or reducing TLR7/8, TLR9, and/or TLR 7/8/9 dependent cell stimulation. As described herein, administration of particular NISCs results in antigen presentation by DCs or APCs while suppressing the TLR 7/8, TLR9, and/ot TLR7/8/9 dependent cell responses associated with immunostimulatory polynucleotides. Such suppression may include decreased levels of one or more TLR-associated cytokines. IRPs appropriate for use in suppressing TLR9 dependent cell stimulation are those IRP that inhibit or suppress cell responses associated with TLR9.

### Methods of Use

The invention provides methods of regulating an immune response in an individual, preferably a mammal, more preferably a human, comprising administering to the individual an antigen-carrier complex as described herein. Methods of immunoregulation provided by the invention include those that suppress and/or inhibit an innate immune response, including, but not limited to, an immune response stimulated by immunostimulatory polypeptides, such as myelin basic protein.

The antigen-carrier complex is administered in an amount sufficient to regulate an immune response. As described herein, regulation of an immune response may be humoral and/or cellular, and is measured using standard techniques in the art and as described herein.

In certain embodiments, the individual suffers from a disorder associated with unwanted immune activation, such as allergic disease or condition, allergy and asthma. An individual having an allergic disease or asthma is an individual with a recognizable symptom of an existing allergic disease or asthma.

In certain embodiments, the individual suffers from a disorder associated with unwanted immune activation, such as autoimmune disease and inflammatory disease. An individual having an autoimmune disease or inflammatory disease is an individual with a recognizable symptom of an existing autoimmune disease or inflammatory disease.

Autoimmune diseases can be divided in two broad categories: organ-specific and systemic. Autoimmune diseases include, without limitation, rheumatoid arthritis (RA), systemic lupus erythematosus (SLE), type I diabetes mellitus, type II diabetes mellitus, multiple sclerosis (MS), immune-mediated infertility such as premature ovarian failure, scleroderma, Sjogren's disease, vitiligo, alopecia (baldness), polyglandular failure, Grave's disease, hypothyroidism, polymyositis, pemphigus vulgaris, pemphigus foliaceus, inflammatory bowel disease including Crohn's disease and ulcerative colitis, autoimmune hepatitis including that associated with hepatitis B virus (HBV) and hepatitis C virus (HCV), hypopituitarism, graft-versus-host disease (GvHD), myocarditis, Addison's disease, autoimmune skin diseases, uveitis, pernicious anemia, and hypoparathyroidism.

Autoimmune diseases may also include, without limitation, Hashimoto's thyroiditis, Type I and Type II autoimmune polyglandular syndromes, paraneoplastic pemphigus, bullus pemphigoid, dermatitis herpetiformis, linear IgA disease, epidermolysis bullosa acquisita, erythema nodosa, pemphigoid gestationis, cicatricial pemphigoid, mixed essential cryoglobulinemia, chronic bullous disease of childhood, hemolytic anemia, thrombocytopenic purpura, Goodpasture's syndrome, autoimmune neutropenia, myasthenia gravis, Eaton-Lambert myasthenic syndrome, stiff-man syndrome, acute disseminated encephalomyelitis, Guillain-Barre syndrome, chronic inflammatory demyelinating polyradiculoneuropathy, multifocal motor neuropathy with conduction block, chronic neuropathy with monoclonal gammopathy, opsonoclonus-myoclonus syndrome, cerebellar degeneration, encephalomyelitis, retinopathy, primary biliary sclerosis, sclerosing cholangitis, gluten-sensitive enteropathy, ankylosing spondylitis, reactive arthritides, polymyositis/dermatomyositis, mixed connective tissue disease, Bechet's syndrome, psoriasis, polyarteritis nodosa, allergic anguitis and granulomatosis (Churg-Strauss disease), polyangiitis overlap syndrome, hypersensitivity vasculitis, Wegener's granulomatosis, temporal arteritis, Takayasu's arteritis, Kawasaki's disease, isolated vasculitis of the central nervous system, thromboangiutis obliterans, sarcoidosis, glomerulonephritis, and cryopathies. These conditions are well known in the medical arts and are described, for example, in Harrison's Principles of Internal Medicine, 14th ed., Fauci A S et al., eds., New York: McGraw-Hill, 1998.

In some embodiments, the invention relates to uses of compositions of this invention prior to the onset of disease. In other embodiments, the invention relates to uses of the compositions of this invention to inhibit ongoing disease. In some embodiments, the invention relates to ameliorating disease in a subject. By ameliorating disease in a subject is meant to include treating, preventing or suppressing the disease in the subject.

In some embodiments, the invention relates to preventing the relapse of disease. For example, an unwanted immune response can occur at one region of a peptide (such as an antigenic determinant). Relapse of a disease associated with an unwanted immune response can occur by having an immune response attack at a different region of the peptide. T-cell responses in some immune response disorders, including MS and other Th1/17-mediated autoimmune diseases, can be dynamic and evolve during the course of relapsing-remitting and/or chronic-progressive disease. The dynamic nature of the T-cell repertoire has implications for treatment of certain diseases, since the target may change as the disease progresses. Previously, pre-existing knowledge of the pattern of responses was necessary to predict the progression of disease. The present invention provides compositions that can prevent the effect of dynamic changing disease, a function of “epitope spreading.” A known model for relapse is an immune reaction to proteolipid protein (PLP) as a model for multiple sclerosis (MS). Initial immune response can occur by a response to PLP₁₃₉₋₁₅₁. Subsequent disease onset can occur by a relapse immune response to PLP₁₇₈₋₁₉₁. Compositions of the present invention have been shown to prevent relapse of disease using the PLP model.

Certain embodiments of this invention relate to priming of immune tolerance in an individual not previously tolerized by therapeutic intervention. These embodiments generally involve a plurality of administrations of a combination of antigen and mucosal binding component. Typically, at least three administrations, frequently at least four administrations, and sometimes at least six administrations are performed during priming in order to achieve a long-lasting result, although the subject may show manifestations of tolerance early in the course of treatment. Most often, each dose is given as a bolus administration, but sustained formulations capable of mucosal release are also suitable. Where multiple administrations are performed, the time between administrations is generally between 1 day and 3 weeks, and typically between about 3 days and 2 weeks. Generally, the same antigen and mucosal binding component are present at the same concentration, and the administration is given to the same mucosal surface, but variations of any of these variables during a course of treatment may be accommodated.

Other embodiments of this invention relate to boosting or extending the persistence of a previously established immune tolerance. These embodiments generally involve one administration or a short course of treatment at a time when the established tolerance is declining or at risk of declining. Boosting is generally performed 1 month to 1 year, and typically 2 to 6 months after priming or a previous boost. This invention also includes embodiments that involve regular maintenance of tolerance on a schedule of administrations that occur semiweekly, weekly, biweekly, or on any other regular schedule.

Other embodiments of this invention relate to treatment of pathological conditions relating to an unwanted hypersensitivity. The hypersensitivity can be any one of types I, II, III, and IV. Immediate (type I) hypersensitivity is typically treated by using one or more offending allergen or tolerogenic fragments thereof as the inducing antigen. The frequency of administration will typically correspond with the timing of allergen exposure. Suitable animal models are known in the art (for example, Gundel et al., Am. Rev. Respir. Dis. 146:369, 1992; Wada et al., J. Med. Chem. 39, 2055, 1996; and WO 96/35418).

Other embodiments of this invention relate to transplantation. This refers to the transfer of a tissue sample or graft from a donor individual to a recipient individual, and is frequently performed on human recipients who need the tissue in order to restore a physiological function provided by the tissue. Tissues that are transplanted include (but are not limited to) whole organs such as kidney, liver, heart, lung; organ components such as skin grafts and the cornea of the eye; and cell suspensions such as bone marrow cells and cultures of cells selected and expanded from bone marrow or circulating blood, and whole blood transfusions.

A serious potential complication of any transplantation ensues from antigenic differences between the host recipient and the engrafted tissue. Depending on the nature and degree of the difference, there may be a risk of an immunological assault of the graft by the host, or of the host by the graft, or both, may occur. The extent of the risk is determined by following the response pattern in a population of similarly treated subjects with a similar phenotype, and correlating the various possible contributing factors according to well accepted clinical procedures. The immunological assault may be the result of a preexisting immunological response (such as preformed antibody), or one that is initiated about the time of transplantation (such as the generation of T_{H} cells). Antibody, T_{H} cells, or T_{C} cells may be involved in any combination with each other and with various effector molecules and cells.

It is an object of this invention to provide materials and procedures that permit transplantation to be conducted according to standard surgical procedures, but with decreased risk of an adverse immunological reaction to the recipient of the transplant. The procedures involve tolerizing the recipient to the tissues of the donor, or vice versa, or both. The tolerizing is performed by administering a target antigen expressed in the transplanted tissue, or a bystander antigen, in a composition of the present invention. The target antigen may be, for example, allogeneic cell extracts. The graft may be a complex structure of many different cell types, and any one or more of the cell types transplanted into the individual may pose a risk for which the procedures of this invention are appropriate. For example, endothelial cell antigens complicate renal transplants, and passenger lymphocytes complicate hepatic transplants.

Certain embodiments of the invention relate to decreasing the risk of host versus graft disease, leading to rejection of the tissue graft by the recipient. The treatment may be performed to prevent or reduce the effect of a hyperacute, acute, or chronic rejection response. Treatment is preferentially initiated sufficiently far in advance of the transplant so that tolerance will be in place when the graft is installed; but where this is not possible, treatment can be initiated simultaneously with or following the transplant. Regardless of the time of initiation, treatment will generally continue at regular intervals for at least the first month following transplant. Follow-up doses may not be required if a sufficient accommodation of the graft occurs, but can be resumed if there is any evidence of rejection or inflammation of the graft. Of course, the tolerization procedures of this invention may be combined with other forms of immunosuppression to achieve an even lower level of risk.

Certain embodiments of this invention relate to decreasing the risk of graft versus host disease. In this series of embodiments, it is necessary to tolerize a living donor against a target antigen of the future graft recipient before the transplantation occurs. Once tolerance is achieved, the cells or tissue of the donor are harvested and the transplant is performed.

Animal models for the study of autoimmune disease are known in the art. For example, animal models which appear most similar to human autoimmune disease include animal strains which spontaneously develop a high incidence of the particular disease. Examples of such models include, but are not limited to, the nonobeses diabetic (NOD) mouse, which develops a disease similar to type 1 diabetes, and lupus-like disease prone animals, such as New Zealand hybrid, MRL-Fas^{lpr} and BXSB mice. Animal models in which an autoimmune disease has been induced include, but are not limited to, experimental autoimmune encephalomyelitis (EAE), which is a model for multiple sclerosis, collagen-induced arthritis (CIA), which is a model for rheumatoid arthritis, and experimental autoimmune uveitis (EAU), which is a model for uveitis. Animal models for autoimmune disease have also been created by genetic manipulation and include, for example, IL-2/IL-10 knockout mice for inflammatory bowel disease, Fas or Fas ligand knockout for SLE, and IL-1 receptor antagonist knockout for rheumatoid arthritis.

### Administration and Assessment of the Immune Response

The carrier can be administered in combination with other pharmaceutical agents, as described herein, and can be combined with a physiologically acceptable carrier thereof (and as such the invention includes these compositions). The carrier may be any of those described herein.

Compositions of this invention can be prepared for administration to an individual in need thereof, particularly human subjects having an unwanted immune response. The preparation of compositions and their use is conducted in accordance with generally accepted procedures for the preparation of pharmaceutical compositions.

Procedures for preparing pharmaceutical compositions are described in Remington's Pharmaceutical Sciences, E. W. Martin ed., Mack Publishing Co., Pa. The mucosal binding component and the antigen (whether given separately or together) are optionally combined with other active components, carriers and excipients, and stabilizers. Additional active components of interest are agents that enhance the tolerogenic effect of the combination at the mucosal surface. An example of an additional active component is a cytokine, exemplified by IL-4. Although not required, pharmaceutical compositions can be supplied in unit dosage form suitable for administration of a precise amount.

The effective amounts and method of administration of the present invention for modulation of an immune response can vary based on the individual, what condition is to be treated and other factors evident to one skilled in the art. Factors to be considered include route of administration and the number of doses to be administered. Such factors are known in the art and it is well within the skill of those in the art to make such determinations without undue experimentation. A suitable dosage range is one that provides the desired regulation of immune response (e.g., suppression of IFN-α or other cytokine production). Useful dosage ranges of the carrier, given in amounts of carrier delivered, may be, for example, from about any of the following: 0.5 to 10 mg/kg, 1 to 9 mg/kg, 2 to 8 mg/kg, 3 to 7 mg/kg, 4 to 6 mg/kg, 5 mg/kg, 1 to 10 mg/kg, 5 to 10 mg/kg. Alternatively, the dosage can be administered based on the number of particles. For example, useful dosages of the carrier, given in amounts of carrier delivered, may be, for example, from about any of the following: greater than 10⁶, 10⁷, 10⁸, 10⁹, or 10¹⁰ particles per dose, or from 1×10⁷ to 1×10⁹ particles per dose, or from 1×10⁸ to 1×10⁹ particles per dose, or from 2×10⁹ to 5×10⁹ particles per dose. The absolute amount given to each patient depends on pharmacological properties such as bioavailability, clearance rate and route of administration. Details of pharmaceutically acceptable carriers, diluents and excipients and methods of preparing pharmaceutical compositions and formulations are provided in Remmingtons Pharmaceutical Sciences 18^{th} Edition, 1990, Mack Publishing Co., Easton, Pa., USA., which is hereby incorporated by reference in its entirety.

The effective amount and method of administration of the particular carrier formulation can vary based on the individual patient, desired result and/or type of disorder, the stage of the disease and other factors evident to one skilled in the art. The route(s) of administration useful in a particular application are apparent to one of skill in the art. Routes of administration include but are not limited to topical, dermal, transdermal, transmucosal, epidermal, parenteral, gastrointestinal, and naso-pharyngeal and pulmonary, including transbronchial and transalveolar. A suitable dosage range is one that provides sufficient IRP-containing composition to attain a tissue concentration of about 1-50 μM as measured by blood levels. The absolute amount given to each patient depends on pharmacological properties such as bioavailability, clearance rate and route of administration.

The present invention provides carrier formulations suitable for topical application including, but not limited to, physiologically acceptable implants, ointments, creams, rinses and gels. Exemplary routes of dermal administration are those which are least invasive such as transdermal transmission, epidermal administration and subcutaneous injection.

Transdermal administration is accomplished by application of a cream, rinse, gel, etc. capable of allowing the carrier to penetrate the skin and enter the blood stream. Compositions suitable for transdermal administration include, but are not limited to, pharmaceutically acceptable suspensions, oils, creams and ointments applied directly to the skin or incorporated into a protective carrier such as a transdermal device (so-called “patch”). Examples of suitable creams, ointments etc. can be found, for instance, in the Physician's Desk Reference. Transdermal transmission may also be accomplished by iontophoresis, for example using commercially available patches which deliver their product continuously through unbroken skin for periods of several days or more. Use of this method allows for controlled transmission of pharmaceutical compositions in relatively great concentrations, permits infusion of combination drugs and allows for contemporaneous use of an absorption promoter.

Parenteral routes of administration include but are not limited to electrical (iontophoresis) or direct injection such as direct injection into a central venous line, intravenous, intramuscular, intraperitoneal, intradermal, or subcutaneous injection. Formulations of carrier suitable for parenteral administration are generally formulated in USP water or water for injection and may further comprise pH buffers, salts bulking agents, preservatives, and other pharmaceutically acceptable excipients. Immunoregulatory polynucleotide for parenteral injection may be formulated in pharmaceutically acceptable sterile isotonic solutions such as saline and phosphate buffered saline for injection.

Gastrointestinal routes of administration include, but are not limited to, ingestion and rectal routes and can include the use of, for example, pharmaceutically acceptable powders, pills or liquids for ingestion and suppositories for rectal administration.

Naso-pharyngeal and pulmonary administration include are accomplished by inhalation, and include delivery routes such as intranasal, transbronchial and transalveolar routes. The invention includes formulations of carrier suitable for administration by inhalation including, but not limited to, liquid suspensions for forming aerosols as well as powder forms for dry powder inhalation delivery systems. Devices suitable for administration by inhalation of carrier formulations include, but are not limited to, atomizers, vaporizers, nebulizers, and dry powder inhalation delivery devices.

As is well known in the art, solutions or suspensions used for the routes of administration described herein can include any one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

As is well known in the art, pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and can be fluid to the extent that easy syringability exists. It can be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Some embodiments include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

As is well known in the art, sterile injectable solutions can be prepared by incorporating the active compound(s) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Certain embodiments of this invention relate to kits and reagents in which one or more component is provided in a separate container, optionally with written instructions, for assembly of a pharmaceutical composition by the patient or the administering health professional.

### EXAMPLES

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1

### Induction of Tolerance to EAE Relapse Using PLP Peptides Conjugated to Polystyrene Spheres

Polystyrene microspheres were coupled either with encephalitogenic epitopes or control peptides to determine whether active EAE can be induced using artificial carriers.

Methods for determination of inhibition of induced EAE followed procedures as previously described (Smith and Miller (2006) J. Autoimmun. 27:218-31; Turley and Miller (2007) J Immunol. 178:2212-20). Briefly, SJL mice, 6-7 weeks old, were purchased from Harlan Laboratories, Bethesda, MD. All mice were housed under specific pathogen-free conditions (SPF) in the Northwestern University Center for Comparative Medicine. Paralyzed animals were afforded easier access to food and water.

Fluoresbrite YG Carboxylate microspheres were purchased from Polysciences, Inc. (Warrington, Pa.). Synthetic peptides PLP₁₃₉₋₁₅₁ (HSLGKWLGHPDKF) and OVA₃₂₃₋₃₃₉ (ISQAVHAAHAEINEAGR) were purchased from Genemed, San Francisco, Calif. The peptides were coupled to the microspheres using ECDI to a specific number of active amino or carboxyl sites on the particles.

Polystyrene microsphere suspensions were coupled with peptides using ethylene carbodiimide (ECDI). Microspheres were washed 2× in PBS, resuspended at 3.2×10⁶/m1 in PBS with 1 mg/ml of each peptide and 30.75 mg/ml ECDI (CalBiochem, La Jolla, Calif.), and incubated for 1 h at 4° C. with periodic shaking. Peptide-coupled microspheres were then washed 3× in PBS, filtered through a 70 μm cell strainer, and resuspended at 250×10⁶ microspheres/ml in PBS. Female mice 8-10 weeks old were injected intravenously with 10⁹ of the indicated Fluoresbrite Carboxylate YG 0.50 micron microspheres coupled with PLP₁₃₉₋₁₅₁ or OVA₃₂₃₋₃₃₉ on day −7 relative to priming with PLP₁₃₉₋₁₅₁/Complete Freund's Adjuvant (CFA) on day 0. Individual animals were observed every 1-3 days, and clinical scores were assessed on a scale of 0-4 as follows: 0=no abnormality; 1=limp tail or hind limb weakness; 2=limp tail and hind limb weakness; 3=partial hind limb paralysis; 4=complete hind limb paralysis. Data are reported as the mean daily clinical score. Mice were observed for clinical signs of EAE for an additional 40 days.

Mice were anesthetized and perfused with 30 ml PBS on the indicated days post-immunization. Spinal cords were removed by dissection, and 2- to 3-mm spinal cord blocks were immediately frozen in OCT (Miles Laboratories; Elkhart, IN) in liquid nitrogen. The blocks were stored at −80° C. in plastic bags to prevent dehydration. Six micrometer thick cross-sections from the lumbar region (approximately L2-L3) were cut on a Reichert-Jung Cyocut CM1850 cryotome (Leica, Deerfield, Ill.), mounted on Superfrost Plus electrostatically charged slides (Fisher, Pittsburgh, Pa.), air dried, and stored at −80° C. Slides were stained using a Tyramide Signal Amplification (TSA) Direct kit (NEN, Boston, Mass.) according to manufacturer's instructions. Lumbar sections from each group were thawed, air-dried, fixed in 2% paraformaldehyde at room temperature, and rehydrated in 1×PBS. Nonspecific staining was blocked using anti-CD16/CD32, 2.4G2; BD PharMingen), and an avidin/biotin blocking kit (Vector Laboratories) in addition to the blocking reagent provided by the TSA kit. Tissues were stained with biotin-conjugated Abs anti-mouse CD4 (H129.19) (BD Biosciences, San Jose, Calif.) and anti-mouse F4/80 (BM8) (Caltag, Burlingame, Calif.). Sections were coverslipped with Vectashield mounting medium including DAPI (Vector Laboratories, Burlingame, Calif.). Slides were examined and images were acquired via epifluorescence using the SPOT RT camera (Diagnostic Instruments, Sterling Heights, Mich.) and Metamorph imaging software (Universal Imaging, Downingtown, Pa.). Eight non-serial lumbar sections from each sample per group were analyzed at 100× and 200× magnification.

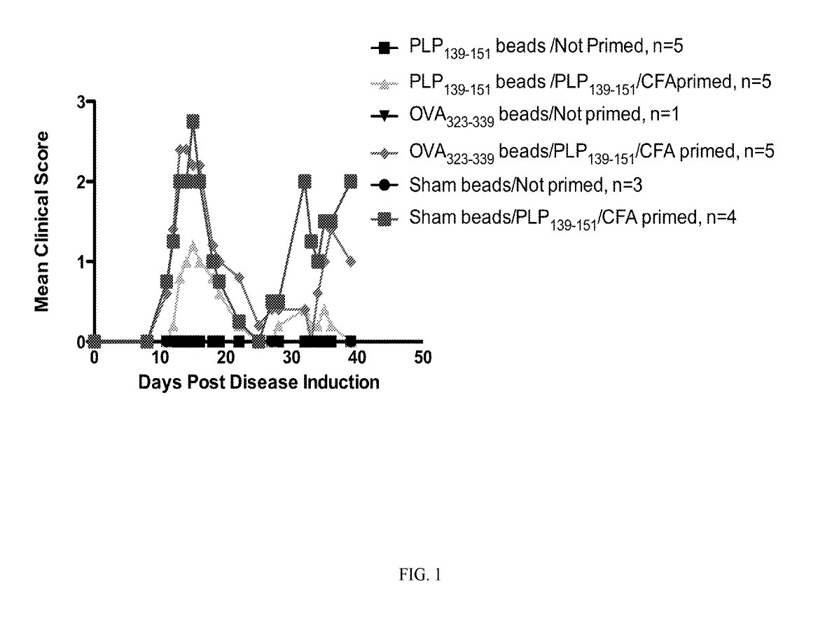

The results are shown in FIG. 1. Fluoresbrite Carboxylate YG 0.50 micron polystyrene Microspheres (Polysciences, Warrington, Pa.) coupled with PLP₁₃₉₋₁₅₁ via an amino linkage, but not with OVA₃₂₃₋₃₃₉, were not only effective in providing significant protection from induction of PLP₁₃₉-151/CFA-induced EAE but more importantly completely abrogated the initiation of relapse of active EAE in SJL mice. This may indicate that inclusion of an apoptotic signal on the inert carrier may not be necessary depending on the composition of the carrier beads.

### Example 2

### Formulation of PLG Microspheres

This example describes the formulation of poly(lactide-co-glycolide-) (PLG) microspheres suitable for encapsulating and delivering antigen-specific peptides. The microspheres are prepared using a double emulsion technique (J. H. Eldridge et al. Mol Immunol, 28:287-294, 1991; S. Cohen et al. Pharm Res, 8:713-720, 1991). RG502H is used as the polymer, and polyvinyl alcohol is used as a stabilizer. Encapsulation efficiency is found to increase with increasing PLG concentration in the organic phase (dichloromethane) (30-200 mg/ml), which also correlates with an increase in median microsphere diameter (about 1 to about 10 μm).

### Example 3

### Preparation of Liposomal Composition Containing Myelin Basic Protein

An optimal myelin basic protein (MBP)/liposome ratio is determined empirically by methods previously described (17). To prepare the MBP/lipid combination, the components are first brought to room temperature. The lipid [1,2-dilauroyl-sn-glycero-3-phosph-ocholine (DLPC); Avanti Polar-Lipids, Inc., Alabaster, Ala.] at a concentration of 120 mg/ml is dissolved in tertiary-butanol (Fisher Scientific, Houston, Tex.) then sonicated to obtain a clear solution. MBP at 40 nag/ml is also dissolved in tertiary-butanol and vortexed until all solids are dissolved. The two solutions are then combined in equal amounts (v:v) to achieve the desired ratio of MBP/liposome, mixing by vortexing, frozen at −80° C. for 1-2 h, and lyophilizing overnight to a dry powder prior to storing at −20° C. until needed. Each treatment vial contains 75 mg MBP.

### Example 4

### Synthesis of Poly(Glu-Lys) Polymer

A polypeptide polymer suitable for use as a linking carrier is poly(glutamic acid-lysine) (poly(glutamyl-lysine) or poly(EK)). N-α-Fmoc glutamic acid y-benzyl ester (Fmoc-Glu(OBzl)-OH) is coupled to N-E-CBZ lysine t-butyl ester (H-Lys(Z)-tBu) (both reagents are commercially available from Calbiochem-Novabiochem, San Diego, Calif.) using diisopropylcarbodiimide and 1-hydroxybenzotriazole. The resulting dipeptide, Fmoc-Glu(OBzl)-Lys(Z)-tBu, can be deprotected using piperidine followed by 95% trifluoroacetic acid to yield H-Glu(OBzl)-Lys(Z)-OH. The dipeptide unit can then be freely polymerized to form a mixture of varying chain lengths, by carbodiimide or other condensation. Alternatively, if a defined length is desired, deprotection of the amino terminal with piperidine to afford H-Glu(OBzl)-Lys(Z)-OtBu and deprotection of the carboxyl terminal with 95% trifluoroacetic acid to afford Fmoc-Glu(OBzl)-Lys(Z)-OH enables condensation of the two dipeptides with carbodiimides to give Fmoc-Glu(OBzl)-Lys(Z)-Glu(OBzl)-Lys-(Z)-OtBu. Repetition of this cycle can give poly(Glu(OBzl)-Lys(Z)) of a defined length. For either the random polymer or the defined-length polymer, the benzyl protecting group on glutamic acid and the CBZ protecting group on lysine can be removed simultaneously using either H₂/Pd or strong acids such as liquid HF or trifluoromethanesulfonic acid. This makes available both free amino and free carboxyl groups for use in attaching antigen-specific peptides and/or apoptotic signaling molecules. The free amino groups can be reprotected with Boc, Bpoc or Fmoc groups in order to prevent reaction during derivatization of the carboxylate groups, by using standard methods in the field of peptide chemistry.

### Example 5

### Antigen-Specific Peptide-Conjugated Polymerized Liposomes

Antigen-specific peptides are conjugated to polymerized liposomes to form antigen-specific peptide-conjugated polymerized liposomes for use in induction of tolerance to the antigen-specific peptide.

Lipid components of: 60% pentacosadiynoic acid filler lipid, 29.5% chelator lipid, 10% amine terminated lipid and 0.5% biotinylated lipid are combined in the indicated amounts and the solvents are evaporated. Water is added to yield a solution that is 30 mM in acyl chains The lipid/water mixture is sonicated for at least one hour. During sonication, the pH of the solution is maintained between 7 and 8 with NaOH and the temperature maintained above the gel-liquid crystal phase transition point by the heat generated by sonication. The liposomes are transferred to a petri dish resting on a bed of wet ice and irradiated at 254 nm for at least one hour to polymerize. The polymerized liposomes are collected after passage through a 0.2 μm filter. To form the antigen-specific peptide-conjugated polymerized liposomes, 2.3 μg avidin is combined with 14.9 μg biotinylated antibody in phosphate buffered saline in about 1:3 molar ratio and incubated at room temperature for 15 minutes. This solution is combined with 150 μL of the above formed polymerized liposomes and incubated at 4° C. overnight to form the antigen-specific peptide-conjugated polymerized liposomes.

### Example 6

### Method of Producing Nanoparticles

Nanoparticles are synthesized by inverse emulsion polymerization. An emulsion is created by adding the PEG block copolymer emulsifier, PLURONIC F-127 (Sigma-Aldrich, Buchs, Switzerland) and the monomer propylene sulfide to ultrapure milliQ water under constant stirring. The protected initiator pentaerythritol tetrathioester is deprotected by mixing it with 0.20 mL of 0.5 M sodium methylate solution under stirring for 10 min. Following deprotection, the initiator is then added to the monomer emulsion and 5 min later 60 μl of the base diaza[5.4.0]bicycycloundec-7-ene (DBU) is added to the reaction and allowed to stir continuously for 24 h under an inert atmosphere. The nanoparticles are then exposed to air in order to produce disulfide cross-linking.

The nanoparticles are purified from remaining monomers, base, or free PLURONIC by 2 days of repeated dialysis with a 12-14 kDa MWCO membrane (Spectrum Laboratories, Rancho Dominguez, Calif.) against ultrapure milliQ water. The nanoparticle size distributions are determined by the use of a dynamic light scattering instrument (Malvern, Worcestershire, United Kingdom).

### Example 7

### Myelin Basic Protein Conjugation to Nanoparticles

Antigen conjugation to nanoparticles can be accomplished by functionalizing Pluronic (a block co-polymer of PEG and PPG) surface with proteins or peptides. A conjugation scheme is presented in this example for the protein antigen myelin basic protein (MBP) using a free cysteine residue for chemical conjugation. Other functionalities can be used in related schemes, such as amines at the N-terminus or on lysine residues. Antigen may also be adsorbed to nanoparticle surfaces.

For conjugation of MBP to nanoparticles, Pluronic divinylsulfone is synthesized to which MPB is coupled via a free thiol group on MPB in a Michael addition reaction. Synthetic details for both steps are given below.

Pluronic F127 (Sigma), divinylsulfone (Fluka), sodium hydride (Aldrich), toluene (VWR), acetic acid (Fluka), diethylether (Fisher), dichloromethane (Fisher) and Celite (Macherey Nagel) are used as received. The reaction is carried out under argon (Messer). ¹H NMR is measured in deuterated chloroform (Armar) and chemical shifts are given in ppm relative to internal standard tetramethylsilane (Armar) signal at 0.0 ppm.

A solution of Pluronic F-127 in toluene is dried by azeotropic distillation using a Dean-Stark trap. The solution is cooled in an ice bath sodium hydride is added. The reaction mixture is stirred for 15 min and divinyl sulfone (Sigma-Aldrich) is added quickly. After stirring in the dark for 5 days at room temperature the reaction is quenched by adding acetic acid. After filtering over celite and concentrating the filtrate under reduced pressure to a small volume the product is precipitated in 1 liter of ice-cold diethylether. The solid is filtered off, dissolved in a minimum amount of dichloromethane and precipitated in ice-cold diethylether four times in total. The polymer is dried under vacuum and stored under argon at −20° C.

### Example 8

### Flow Cytometry & Analysis and In Vitro Nanoparticle Internalization: Uptake by APCs, Including DCS

Flow cytometry analysis is performed to quantify the fraction of APCs and DCs in lymph nodes that are internalizing nanoparticles. Following staining, lymph node cell suspensions are analyzed by flow cytometry (CyAn ADP, Dako, Glostrup, Denmark). Further analysis is performed using FlowJo software (TreeStar, Ashland, Oreg.). APCs and DCs with internalized fluorescent nanoparticles are determined to be MHCII⁺FITC⁺ and CD11c⁺FITC⁺, respectively, FITC representing labeling of the nanoparticles. DC maturation following nanoparticle internalization is evaluated by calculating the fraction of cells that expressed CD86 and CD80.

### Example 9

### Modification of Quantum Dot Nanoparticle

The amine surface of the quantum dot nanoparticle including dendrimer-encapsulated quantum dots is modified with a linking agent such as Sulfo-SMCC (Sulfosuccinimidyl 4-N-maleimidomethyl cyclohexane-1-carboxylate) to form a maleimide-activated quantum dot. Any excess reagent is removed by size-exclusion chromatography, a commonly-employed technique to separate the components of the reaction mixture. Methods for encapsulating quantum dots with dendrimers are described in, for example, Lemon et al. (J. Am. Chem. Soc., 2000, 122:12886), the contents of which are incorporated herein by reference.

Formation of Quantum Dot Nanoparticle-AChE Conjugate

Maleimide-activated QD is reacted with the sulfhydryl-modified AChE for a limited time to prevent multiple QDs from attaching to multiple proteins. Multiple crosslinking is not favored due to steric hinderance and entropy of large molecules. The reaction is halted by separation using size-exclusion chromatography.

### Example 10

### Method of Producing Dendrimers

PAMAM dendrimers are composed of an ethylenediamine (EDA) initiator core with four radiating dendron arms, and are synthesized using repetitive reaction sequences comprised of exhaustive Michael addition of methyl acrylate (MA) and condensation (amidation) of the resulting ester with large excesses of EDA to produce each successive generation. Each successive reaction therefore theoretically doubles the number of surface amino groups, which can be activated for functionalization. The synthesized dendrimer has been analyzed and the molecular weight has been found to be 26,380 g/mol by GPC and the average number of primary amino groups has been determined by potentiometric titration to be 110.

### Example 11

### Characterization of Dendrimer Functional Groups

Acetylation of the dendrimer.

Acetylation is the first requisite step in the synthesis of dendrimers. Partial acetylation is used to neutralize a fraction of the dendrimer surface from further reaction or intermolecular interaction within the biological system, therefore preventing non-specific interactions from occurring during synthesis. Leaving a fraction of the surface amines non-acetylated allows for attachment of functional groups. Acetylation of the remaining amino groups results in increased water solubility (after FITC conjugation), allowing the dendrimer to disperse more freely within aqueous media with increased targeting specificity, as compared to many conventional mediums (Quintana et al., Pharm. Res. 19, 1310 (2002)).

### Example 12

### Conjugation of Functional Groups to Acetylated Dendrimer

Conjugation of fluorescein isothiocyanate to acetylated dendrimer. A partially acetylated PAMAM dendrimer is used for the conjugation of fluorescein isothiocyanate (FITC) in order to increase the solubility of the dendrimer. The partially acetylated dendrimer is allowed to react with fluorescein isothiocyanate, and after intensive dialysis, lyophilization and repeated membrane filtration the dendrimer-FITC product is yielded.

Conjugation of folic acid to acetylated dendrimer.

Conjugation of folic acid to the partially acetylated mono-functional dendritic device is carried out via condensation between the γ-carboxyl group of folic acid and the primary amino groups of the dendrimer. This reaction mixture is added drop wise to a solution of DI water containing dendrimer-FITC and vigorously stirred for 2 days (under nitrogen atmosphere) to allow for the folic acid to fully conjugate to the dendrimer-FITC.

### Example 13

### Evaluating Tolerance by T Cell Phenotypic Analysis

A nanoparticle-MBP(83-99) complex of the invention is dissolved in phosphate-buffered saline (PBS) and injected into Female Lewis rats intraperitoneally in 0A-0.2 ml containing 500 μg of the nanoparticle-MBP complex. The control group of rats receives 0.1-0.2 ml of PBS. Nine to ten days after the injection, spleen and lymph nodes (inguinal and lumbar) are harvested from the rats and single cell suspensions obtained by macerating tissues through a 40 μm nylon cell strainer. Samples are stained in PBS (1% FCS) with the appropriate dilution of the relevant monoclonal antibodies. Propidium iodide staining cells are excluded from analysis. Samples are acquired on an LSR2 flow cytometer (BD Biosciences, USA) and analysed using FACS Diva software. The expression of the activation markers CD25, CD44, CD62L, CTLA-4, CD45Rb, and CD69 is analyzed on splenocytes and lymph node cells from the rats injected with the nanoparticle-MBP complex. The CD4⁺ T cells from the complex-injected rats express a CD25^{hi}/CD45RB^{int} phenotype, which is characteristic of anergized CD4⁺ T cells. There is also a higher percentage of CD4⁺ cells that is CD25^{hi} and FoxP3⁺, suggesting the induction of regulatory T cells.

To assess apoptosis, CD8⁺ T cells are isolated from splenocytes using a CD8α, isolation kit (Miltenyi Biotec, Germany) according to the manufacturer's protocol, stained with annexin V and PI (both BD Biosciences) according to the manufacture's protocol and then analyzed by flow cytometry. For intracellular staining of Granzyme B and Bcl-2, T cells are permeabilized with BD cytofix/cytoperm kit (BD Biosciences) and stained with rat-anti-mouse Granzyme B PE mAb (eBioscience) or hamster-anti-mouse Bcl-2 FITC mAb (BD Biosciences). Specificity controls are performed using the appropriate isotype mAbs. Samples are analyzed by flow cytometry, which is well known in the art.

### Example 14

### Evaluating Tolerance by T Cell Proliferation

A nanoparticle-MBP(83-99) complex of the invention is dissolved in phosphate-buffered saline (PBS) and injected into Balb/c mice intraperitoneally in 0.1-0.2 ml containing 500 μg of the nanoparticle-MBP complex. The control group of mice receives 0.1-0.2 ml of PBS. Nine to ten days after the injection, spleen and lymph nodes (inguinal and lumbar) are harvested from the mice and single cell suspensions obtained by macerating tissues through a 40 μm nylon cell strainer. CD4⁺ T cells are isolated from splenocytes using a CD4⁺ T cell isolation kit (Miltenyi Biotec, Germany) according to the manufacturer's protocol. Purified CD4⁺ T cells are plated (5×10⁴/well) in triplicate with 1) T cell-depleted, irradiated (2000 R) CBA/J stimulators (5×10⁵) for 72 hr, 2) syngeneic splenocytes (5×10⁵) plus soluble anti-CD3 (145-2C11) for 48 hr, or 3) 100 ng/ml phorbol 12-myristate β-acetate (Sigma) and 200 nM calcium ionophore (ionomycin, Sigma) for 36 hr total, with a pulse of 1 μCi/well [³H]TdR during the last 8-12 hr. [³H]TdR incorporation as an indicator of DNA replication and cell proliferation is measured in the presence of scintillation fluid on a β-counter (Beckman Coulter, Inc., Fullerton, Calif.). Supernatants from anti-CD3-stimulated T cells are harvested at 48 hr, and the production of IL-2 is determined by measuring proliferation of the IL-2-dependent cell line CTLL-2 in the presence of anti-IL-4 antibody (11B11), where 1 unit is the amount of IL-2 required to support half-maximal [³11]TdR incorporation.

T cell proliferation may also be assessed by directly visualizing the division of cells using the fluorescent cytosolic dye, Carboxyfluorescein Diacetate Succinimidyl Ester (CFSE). For CSFE labeling of cells, splencoytes from the mice injected with the nanoparticle-MBP complex are incubated with 3 μM CFSE (Molecular Probes, UK) for 3 min at 37° C. in 1 ml of PBS. In each well of a 96 well-plate, 2×10⁵ CFSE-labeled splenocytes are stimulated with 1×10⁵ irradiated (80 Gy) temperature-induced RMA-S coated with MBP(83-99) peptide (10 μM) or an irrelevant control peptide, pSV9 (10 μM). The appropriate cultures are supplemented with 10 U/ml IL-2, 10 ng/ml IL-7, 50 ng/ml IL-15 or 50 ng/ml IL-21. Cells are harvested at the appropriate time point, stained with CD4 and subjected to CFSE profiling by flow cytometry.

### Example 15

### Evaluating Tolerance by T Cell Cytokine Profile and Cytotoxicity

### IFN-γ Assays

A nanoparticle-MBP(83-99) complex of the invention is dissolved in phosphate-buffered saline (PBS) and injected into Balb/c mice intraperitoneally in 0.1-0.2 ml containing 500 μg of the nanoparticle-MBP complex. The control group of mice receives 0.1-0.2 ml of PBS. Nine to ten days after the injection, spleen and lymph nodes (inguinal and lumbar) are harvested from the mice and single cell suspensions obtained by macerating tissues through a 40 μm nylon cell strainer. To measure antigen specific IFN-γ production, CD4⁺ T cells are isolated from splenocytes using a CD4⁺ T cell isolation kit (Miltenyi Biotec, Germany) according to the manufacturer's protocol. Purified CD4⁺ T cells are stimulated, culture supernatant harvested and IFN-γ measured in an IFN-γ ELISA. Triplicate cultures are plated for each different experimental condition on 96-well round-bottom plates. In each well 1×10⁴ splenocytes from the mice injected with the nanoparticle-MBP(83-99) complex are stimulated with 1×10⁴ irradiated (80 Gy) temperature-induced RMA-S coated with MBP(83-99) peptide (10 μM) or an irrelevant control peptide, pSV9. The appropriate cultures are supplemented with 10 U/ml IL-2, 10 ng/ml IL-7, 50 ng/ml IL-15 or 50 ng/ml IL-21. After 72 hours, 50 μl culture supernatant is harvested from each well and murine IFN-γ is measured by sandwich ELISA using anti-IFN-γ antibodies (BD Biosciences). The activity in experimental samples is ascertained using the standard curve of mean absorbance values (OD) versus the dilution of recombinant IFN-γ in the supernatant.

### IL-2 Bioassays

To measure antigen specific IL-2 production, purified CD4⁺ T cells from the mice injected with the nanoparticle-MBP(83-99) complex are stimulated, culture supernatant harvested and IL-2 measured using IL-2 dependent CTLL cells. The stimulation phase of the IL-2 assay is performed exactly as the IFN-γ assay. After 72 hours, 50 μl culture supernatant is harvested, transferred to wells containing CTLL cells (5×10³) and incubated for 16-18 hours. The cells are pulsed with ³[H]-thymidine by adding 1 ³[H]-thymidine to each well and incubating for a further 12 hours. The activity in experimental samples is ascertained using the standard curve of cpm versus the dilution of recombinant IL-2 in the supernatant. Alternatively, murine IL-2 from the supernatant may be measured by sandwich ELISA using anti-IL-2 antibodies (BD Biosciences).

CTL assays

Cytotoxic activity of CD8⁺ T cells from the mice injected with the nanoparticle-MBP(83-99) complex is determined in 4-hour ⁵¹chromium-release assays against MBL-2 tumor cells and RMA-S cells coated with MBP(83-99) peptides or MHC class I-binding control peptides. ⁵¹chromium-release assays are well known in the art.

### Cytokine ELISA

Purified CD4⁺ T cells from the mice injected with the nanoparticle-MBP(83-99) complex are stimulated and the culture supernatant is harvested as described hereinabove. Levels of cytokines including IL-1, IL-4, IL-5, IL-6, IL-10, IL-13, TGF-β, and TNF-α are measured by cytokine sandwich ELISA using the standard protocols (BD Biosciences). Increased production of IL-4, IL-5, IL-10 and IL-13 is typically associated with a Th2 response. IL-10 and TGF-β are typically associated with a regulatory T cell response.

### Example 16

### Evaluating Tolerance by T Cell Suppressive Activity

A nanoparticle-MBP(83-99) complex of the invention is dissolved in phosphate-buffered saline (PBS) and injected into Balb/c mice intraperitoneally in 0.1-0.2 ml containing 500 μg of the nanoparticle-MBP complex. The control group of mice receives 0.1-0.2 ml of PBS. Nine to ten days after the injection, spleen and lymph nodes (inguinal and lumbar) are harvested from the mice and single cell suspensions obtained by macerating tissues through a 40 μm nylon cell strainer. To measure T cell suppressive activity, CD4⁺ T cells are isolated from splenocytes using a CD4⁺ T cell isolation kit (Miltenyi Biotec, Germany) according to the manufacturer's protocol. CD4+CD25+ regulatory T cells can be isolated using CD25 microbeads.

Culture the CD4+CD25-responder T cells (Tresp) (3×10⁴) in U-bottom 96-well plates with Treg (CD4+CD25+) from the mice injected with the nanoparticle-MBP(83-99) complex in the presence of soluble 0.5-0.75 μg/ml α-CD3 and 2.5-4 μg/ml α-CD28 for 2-4 days. 3×10⁴ irradiated (3000 Rad) T cell-depleted splenocytes can be added as APCs instead of α-CD28 in the co-culture. Label Tresp cells with CFSE to distinguish them from the Treg cells in the co-culture. Measure proliferation of the CD4+CD25-responder T cells by incorporation of ³H-thymidine for the last 6-16 h of culture or by CFSE dilution. Measure cell death at 0 hr and after 1, 2 and 3 days. Cell death analyses of CFSE+ responders should be performed based on forward scatter or Annexin V and propidium iodide staining. Treg isolated from the mice injected with the nanoparticle-MBP(83-99) complex are capable of suppressing the proliferation of the responding CD4+CD25-T cells.

### Example 17

### Induction of Tolerance with gp39 Peptide In Vivo

A human cartilage (HC) gp-39 (263-275)-specific delayed type hypersensitivity (DTH) assay suitable to monitor tolerance induction with peptide antigens is developed. Immunization of Balb/c mice with HC gp-39 (263-275) in incomplete Freunds adjuvant (IFA) is effective in the induction of a DTH response following challenge with the HC gp-39 (263-275) peptide. This peptide-based DTH system is used to detect modulation of the DTH response by parenteral application of nanoparticles conjugated with HC gp-39 (263-275) peptide. Application of the nanoparticles conjugated with HC gp-39 (263-275) peptide, in a dose-dependent manner, downmodulates the HC gp-39 (263-275) induced DTH response, indicating that the nanoparticles conjugated with HC gp-39 (263-275) peptide can efficiently tolerize a peptide-specific response induced with HC gp-39 (263-275).

### Example 18

### Induction of Tolerance in a HLA:DR2 Transgenic Mouse

An antigen-carrier complex of the present invention, when presented by an MHC molecule, can induce immunological tolerance in a humanized mouse model of multiple sclerosis using a peptide selected from within T-cell epitopes of myelin basic protein (MBP) corresponding to MBP 140-154. The mouse model is transgenic for the human MHC molecule HLA:DR2 (DRB1*1501) (Madsen et al. (1999) Nature Genetics 23:343-347).

The induction of anergy or changes in the CD4+ T-cell population in a mouse following administration of an antigen-carrier complex may be monitored by a reduction in T-cell proliferation when challenged with the antigen in vivo.

### Methods

### Antigens

MBP peptide 140-154 is synthesized using L-amino acids and standard F-moc chemistry on an Abimed AMS 422 multiple peptide synthesizer (Abimed, Langenfeld, Germany). The sequence of MBP peptide 140-154 is GFKGVDAQGTLSKIF. MBP peptide 140-154 is conjugated to the nanoparticles as described herein. Purified Protein Derivative of Mycobacterium tuberculosis (PPD) (Veterinary Laboratories, Addlestone, Surrey) is used at a concentration of 50 μg/ml in lymphocyte proliferation assays.

### Mice and Tolerance Induction

HLA:DR2 transgenic are bred in isolators and housed in a specific pathogen-free facility. Within each treatment group, mice are both age (8-12 weeks) and sex matched. Mice are pre-treated with 100 ug of MBP peptide 140-154 in 25 ul of phosphate buffered saline (PBS) or 25 ul PBS alone intranasally (i.n) on days −8, −6 and −4 prior to immunization on day 0.

Mice are immunized subcutaneously at the base of the tail and hind limb with 100 ul of an emulsion consisting of an equal volume of Complete Freund's Adjuvant (CFA) and PBS containing 200 ug MBP140-154 and 400 ug heat-killed Mycobacterium tuberculosis, strain H37RA (Difco, Detroit, Mich.). A control group of mice, previously treated with PBS i.n., are immunized without peptide.

Intranasal pre-treatment followed by immunization give rise to three groups of mice: Group A is tolerized with PBS and immunized with MBP 140-154 (7 mice); Group B is tolerized with MBP 140-154-nanoparticles and then immunized with the same peptide MBP 140-154 (7 mice); and Group C is both tolerized and immunized with PBS.

### Lymph Node Proliferation Assays

After 10 days, draining popliteal and inguinal lymph nodes are removed aseptically. The nodes are disaggregated, washed and resuspended in X-Vivo 15 medium (BioWhittaker, Maidenhead, UK) supplemented with 5×10⁻⁵ M 2-mercaptoethanol and 4 mM L-glutamine. Cells are plated in triplicate at 5×10⁵ cells well and cultured with or without varying concentrations of MBP peptide 140-154 (1-150 ug/ml) for 72 hours. To check for successful immunization of mice, lymph node cells are plated with PPD (50 ug/ml) as described above. Cultures are pulsed for the final 16 hours with 0.5 uCi [³H]-Thymidine. Cells are harvested and T-cell proliferation is expressed as Stimulation Index (SI): corrected counts per minute (ccpm) antigen containing culture/ccpm culture without antigen.

### Results

Mice that are pre-treated i.n. with PBS and then immunized with MBP peptide 140-154 (Group A) respond to antigenic stimulation when re-challenged with MBP140-154 in a dose dependent manner. With increasing concentration of peptide, the SI, a measure of lymphocyte proliferation, increases from a median of 2.5 to 10. All the mice in this group demonstrate that PBS administered intranasally could not induce tolerance to MBP 140-154. In contrast, intranasal pre-treatment with MBP 140-154-nanoparticle have a profound effect on the proliferative response of lymphocytes stimulated with this peptide. Lymphocytes from Group B mice are unable to respond to any significant degree, even at the high peptide concentration of 150 ug/ml (SI median 3). The marked reduction in proliferation in Group B as compared with Group A is evident. The data shows that the MBP 140-154-nanoparticles of the present invention induce tolerance in lymphocytes from HLA-DR2 mice.

Lymphocytes extracted from mice which have been pre-treated and immunized with PBS (Group C) fail to show any response to MBP 140-154 suggests, although they elicit an excellent response to PPD and are therefore immunized against the PPD antigen. This lack of response to MBP peptide within Group C confirms that the proliferative response seen in Group A is indeed a response to immunization with MBP 140-154, and that the responses to both MBP 140-154 and PPD in control Groups A and C are antigen specific. As the proliferation to MBP 140-154 is antigen specific, induction of tolerance to this molecule is also specific.

### CONCLUSION

An MBP peptide-nanoparticle of the invention, e.g., MBP 140-154, that does not require processing and binds to HLA:DR2 MHC Class II molecules, can induce tolerance when administered intranasally.

### Example 19

### Treatment of EAE with the Antigen-Carrier Complex

### Immunizations and EAE Induction

MBP peptide and peptide analogs are dissolved in phosphate-buffered saline (PBS) and emulsified with an equal volume of incomplete Freund's adjuvant supplemented with 4 mg/ml heat-killed Mycobacterium tuberculosis H37Ra in oil (Difco Laboratories, Inc., Detroit, Mich.). Female Lewis rats are immunized subcutaneously at the base of the tail with 0.1-0.2 ml containing 500 ug of peptide in the emulsion and are monitored for clinical signs daily. EAE is scored on a scale of 0-4, as follows: 0, clinically normal; 1, flaccid tail; 2, hind limb weakness; 3, hind limb paralysis; 4, front and hind limbs affected.

In this system, experimental allergic encephalomyelitis (EAE) is induced in twelve female Lewis rats by injection of MBP(83-99) peptide in complete Freund's adjuvant (CFA) at the base of the tail. Nine days later, rats are divided into two groups of six animals and subcutaneously injected with 13.2 mg/kg of either MBP peptide-dendrimer or a control peptide, sperm whale myoglobin (SWM) (110-121). Animals are monitored daily for disease symptoms and scored in a blinded fashion on a nonlinear ascending scale of 0-4 with increments denoting increasing paralysis. Each individual score is averaged with group cohorts to obtain the mean clinical score.

The disease severity in those animals treated with the MBP peptide-dendrimer is about 50% reduced compared to the control group. The MBP peptide-dendrimer reduces the severity and duration of the disease in this model system.

Although these results clearly demonstrate that MBP peptide-dendrimer inhibits the development of EAE, a murine animal model system of EAE has also been developed. The SJL/J (H-2₅) mouse develops a chronic relapsing form of EAE in response to immunization with MBP(83-99) peptide in the presence of pertussis vaccine. The ability of the MBP peptide (83-99)-dendrimer to inhibit the disease is evaluated.

Groups of 10 animals are injected intraperitoneally weekly for 4 weeks with 20 mg/kg of either a control peptide or the peptide analog. The animals are then monitored for disease over the next 2-3 months. SJL/J mice develop symptoms of EAE beginning around day 20 in the control group that last for approximately 3 weeks. Beginning around day 70, a relapse occurs reaching a mean clinical score of about 1. However, weekly injection with the MBP peptide (83-99)-dendrimer for four weeks not only reduces the level of the disease in the first phase, but also reduces the severity of the relapse.

### Example 20

### Production and Use of Peptide-Coupled Polystyrene Microspheres

### Production of Peptide-Coupled Polystyrene Microspheres

If necessary, warm the carboxyl microparticles, PolyLink Coupling Buffer and PolyLink Wash/Storage Buffer (Polysciences, Inc., Warrington, Pa.) to room temperature. Carboxyl (COOH) microparticles can be used for covalent coupling of proteins by activating the carboxyl groups with water-soluble carbodiimide (ECDI). The carbodiimide reacts with the carboxyl group to create an active ester that is reactive toward primary amines on the protein of interest. Pipet 12.5 mg of microparticles into a 1.5 polypropylene microcentrifuge tube. Pellet the microparticles via centrifugation for 5-10 minutes at approximately 10000×G. Resuspend the microparticle pellet in 0.4 ml of PolyLink Coupling Buffer. Pellet again via centrifugation for 5-10 minutes at approximately 10000×G. Resuspend the microparticle pellet in 0.17 ml of PolyLink Coupling Buffer. Just before use, prepare a 200 mg/ml ECDI solution by dissolving 10 mg PolyLink ECDI in 50 μl PolyLink Coupling Buffer. Add 241 of the ECDI solution to the microparticle suspension. Mix gently end-over-end or briefly vortex. Add protein equivalent (e.g. PLP₁₃₉₋₁₅₁, PLP₁₇₈₋₁₉₁ or OVA₃₂₃₋₃₃₉) to 200-500 μg. Mix gently by pipetting. Incubate for 30-60 minutes at room temperature. Centrifuge mixture for 10 minutes at approximately 10000×G. Save this supernatant for determination of the amount of bound protein. Resuspend microparticle pellet in 1 mL sterile PBS. Centrifuge again at 10000×G.

### Injection of Peptide-Coupled Polystyrene Microspheres

Resuspend in 1 mL of sterile PBS. Pass suspension across a 40 μm mesh strainer to remove clumps of cross-linked particles. Raise volume to 4 mL with sterile PBS (500 micrograms of coupled microspheres is sufficient to dose 20 animals). Inject suspended particles into mice via the lateral tail vein.

### Example 21

### Peptide-Coupled Polystyrene Microspheres Induce Specific Tolerance for Both Prevention and Treatment of PLP-Induced EAE

This example describes the effect of administration of peptide-coupled polystyrene microspheres either prior to, or after induction of PLP₁₃₉₋₁₅₁ induced EAE in mice.

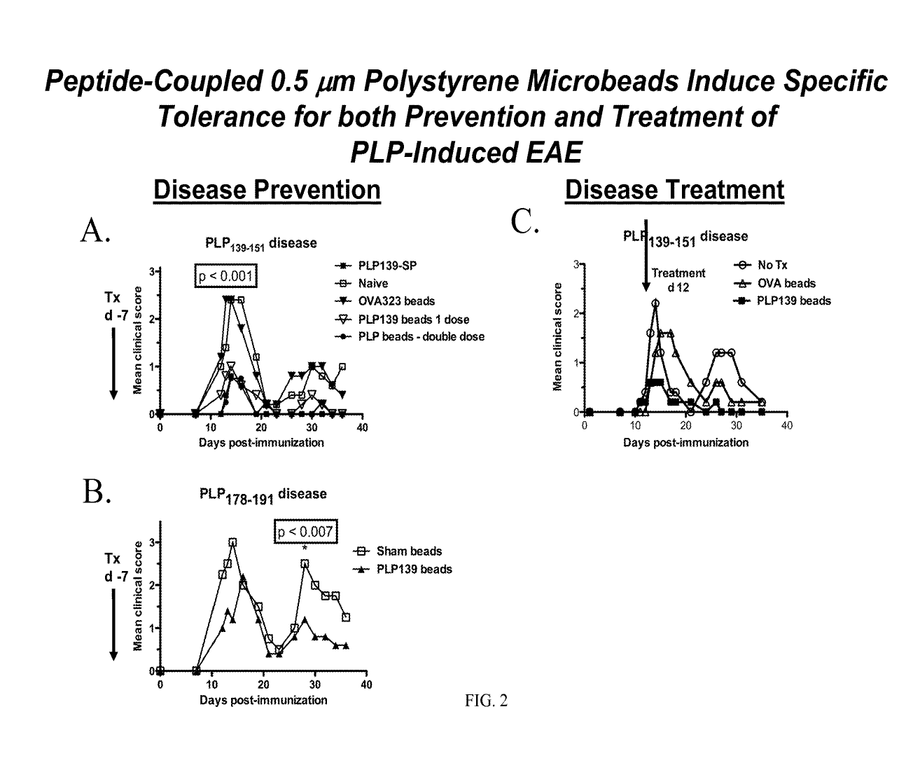

The production of peptide coupled microspheres was performed either as described in Example 1 or Example 20. Either PLP₁₃₉₋₁₅₁ or a control (OVA₃₂₃₋₃₃₉) peptide was coupled to 0.5 μm microspheres. Mice were injected intravenously with either the PLP₁₃₉₋₁₅₁ or control (OVA₃₂₃₋₃₃₉) peptide bound microspheres either on day −7 (“Disease Prevention) or day 12 (“Disease Treatment”) relative to priming with PLP₁₃₉₋₁₅₁ or PLP₁₇₈₋₁₉₁+Complete Freund's Adjuvant (CFA) on day 0. Animals were observed and scored as described in Example 1. The results are shown in FIG. 2. Animals treated prior to disease onset with PLP₁₃₉₋₁₅₁-coated microspheres showed a decrease in clinical score compared to animals treated with Sham beads (microspheres treated with ECDI but no peptide). The results also showed a similar decrease in clinical score to treatment using cells treated to have PLP₁₃₉₋₁₅₁ on the cell surface (see FIGS. 2A and 2B). Animals treated following disease onset with PLP₁₃₉₋₁₅₁-coated microspheres similarly showed a decrease in clinical score compared to animals who were either untreated or treated with microspheres having a control peptide (see FIG. 2C). Therefore, the results show that treatment using peptide-coupled polystyrene microspheres is useful for decreasing disease severity prior to and following disease onset.

### Example 22

### Recall Responses to Primed and Spread Epitopes are Decreased in Tolerized Recipients

This example describes the effect of administration of peptide-coupled polystyrene microspheres on delayed type-hypersensitivity in a mouse model.

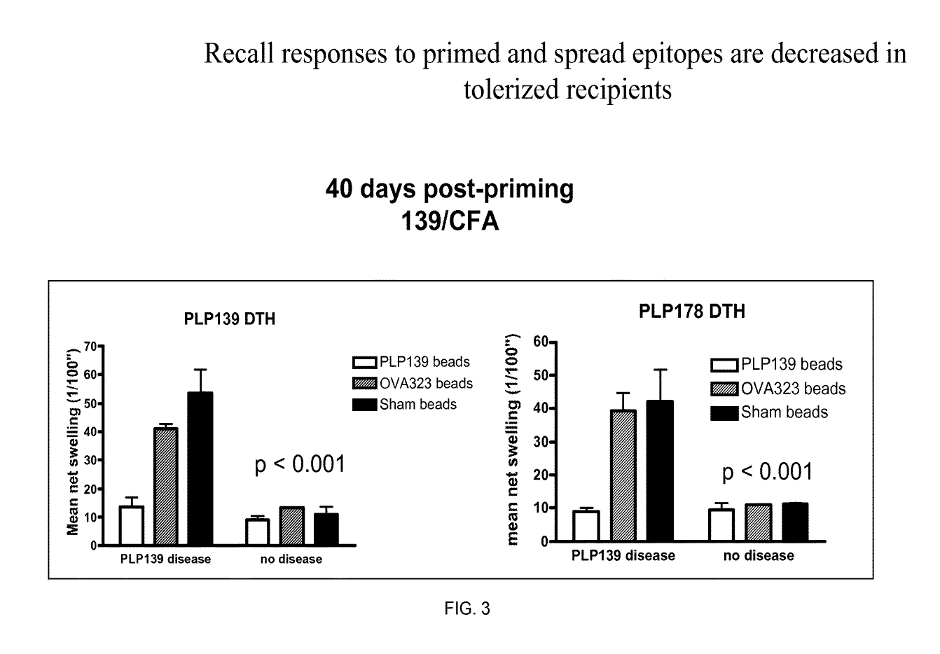

Mice were prepared and treated using either PLP₁₃₉₋₁₅₁, control (OVA₃₂₃₋₃₃₉) peptide, or sham bound microspheres. Recall responses of CD4 T-cells were measured by delayed-type hypersensitivity (DTH) at day 40 relative to priming with PLP₁₃₉₋₁₅₁ or PLP₁₇₈₋₁₉₁/Complete Freund's Adjuvant (CFA) on day 0, as described previously (Smith and Miller (2006) Journal of Autoimmunity 27:218-31; Luo et al. (2008) PNAS 105:14527-32). Measurement of DTH was done by measurement of ear swelling using a 24 hour ear swilling assay. Pre-challenge ear thickness was determined using a Mitutoyo model 7326 engineer's micrometer (Schlesinger's Tools, Brooklyn, N.Y.). Immediately thereafter, DTH responses were elicited by injecting peptide into the dorsal surface of the ear. The increase in ear thickness over pre-challenge measurements was determined 24 hours after ear challenge. Results are shown in FIG. 3. The mean net swelling in mice pre-treated with PLP₁₃₉₋₁₅₁ microspheres was comparable to control, whereas the control (OVA₃₂₃₋₃₃₉) peptide, or sham bound microspheres resulted in increased swelling. These results indicate that microparticles can be used to protect animals from later inflammatory responses.

### Example 23

### Effect of Peptide-Coupled Microspheres on CNS Infiltration

This example describes the effect of administration of peptide-coupled polystyrene microspheres on CNS infiltration of leukocytes into the CNS.

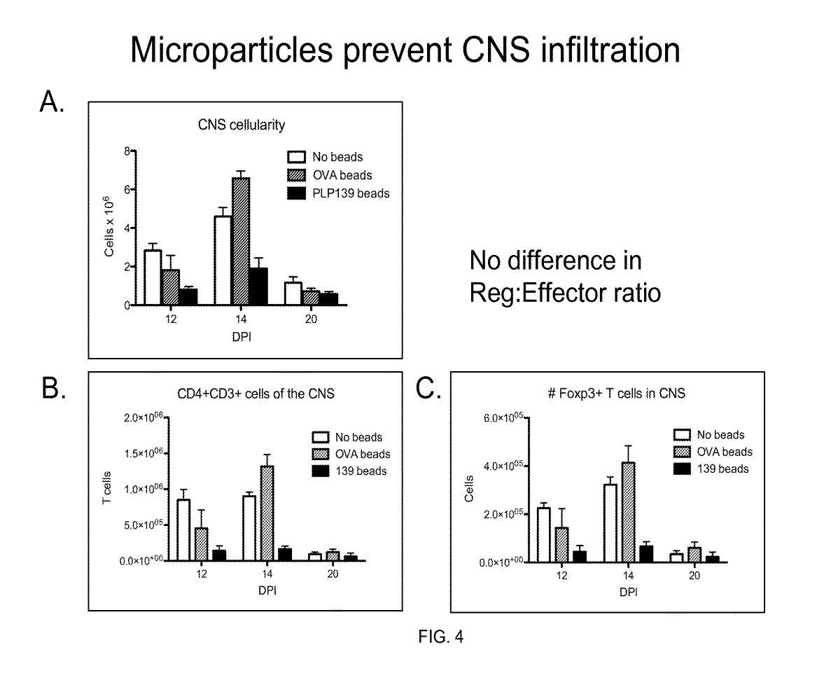

Mice were prepared and treated using either PLP₁₃₉₋₁₅₁, control (OVA₂₃₋₃₃₉) peptide bound microspheres or were not treated with any microspheres. Mice were injected on day −7 relative to priming with PLP₁₃₉₋₁₅₁/Complete Freund's Adjuvant (CFA). Mouse infiltration of leukocytes into the CNS were examined by immunohistochemistry, as described previously (Smith and Miller (2006) Journal of Autoimmunity 27:218-31; Turley and Miller (2007) J Immunol. 178:2212-20). Briefly, mice were anesthetized and perfused with 30 ml PBS on the indicated days post-immunization. Spinal cords were removed by dissection and immediately frozen in liquid nitrogen. Six micrometer thick cross-sections from the lumbar region were cut and mounted on Superfrost Plus electrostatically charged slides (Fisher, Pittsburgh, Pa.), air dried, and stored at −80° C. The results are shown in FIG. 4. Slides were stained for cellularity (FIG. 4A), CD4+CD3+ cells (FIG. 4B) or Foxp3+ cells (FIG. 4C) in the CNS. The results indicate that treatment with PLP₁₃₉₋₁₅₁ treated microspheres resulted in a decrease in leukocytes in the CNS compared to control.

### Example 24

### Effective of Peptide Coupled Microspheres on Splenectomized Animals

This example describes the effect of administration of peptide-coupled polystyrene microspheres on mice that were splenectomized to examine the requirement of spleen activity in the induction of tolerance.

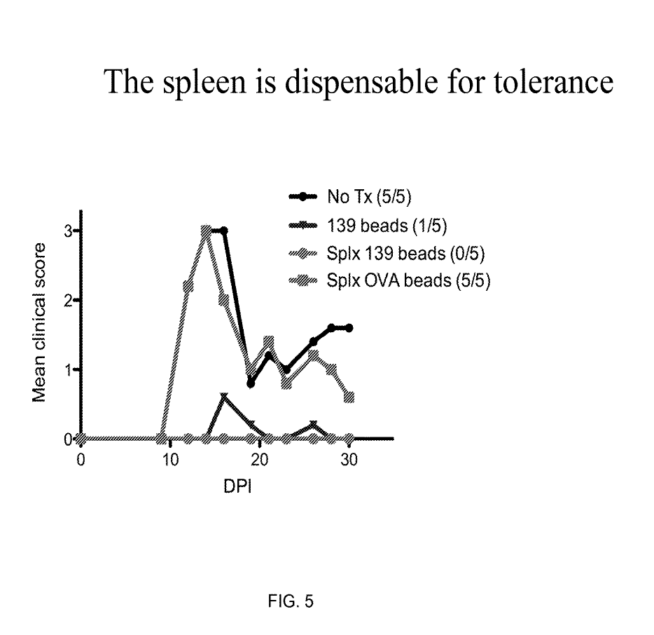

The production of peptide coupled microspheres was performed either as described in Example 1 or Example 20. Splenectomized or intact animals were treated with PLP₁₃₉₋₁₅₁, control (OVA₃₂₃₋₃₃₉) peptide bound microspheres or were not treated with any microspheres. The animals were treated with microspheres on day −7 relative to priming with PLP₁₃₉₋₁₅₁/Complete Freund's Adjuvant (CFA). The results are shown in FIG. 5. The control (OVA₃₂₃₋₃₃₉) peptide bound microspheres showed a mean clinical score similar to animals that were not treated with any microspheres, whereas both mice splenectomized or intact that were treated with PLP₁₃₉₋₁₅₁ microspheres showed a decrease in mean clinical score.

1. A composition for induction of antigen-specific tolerance comprising a carrier particle attached thereto an apoptotic signaling molecule and an antigenic peptide.

2. (canceled)

3. The composition of claim 1, wherein the antigenic peptide is an autoimmune antigen, a transplantation antigen or an allergen.

4. The composition of claim 3, wherein the antigenic peptide is myelin basic protein, acetylcholine receptor, endogenous antigen, myelin oligodendrocyte glycoprotein, pancreatic beta-cell antigen, insulin, glutamic acid decarboxylase (GAD), collagen type 11, human carticlage gp39, fp130-RAPS, proteolipid protein, fibrillarin, small nucleolar protein, thyroid stimulating factor receptor, histones, glycoprotein gp70, pyruvate dehydrogenase dehyrolipoamide acetyltransferase (PCD-E2), hair follicle antigen or human tropomyosin isoform 5.

5. The composition of claim 1, wherein the antigenic peptide is coupled to the carrier by a conjugate molecule.

6. The composition of claim 5, wherein the conjugate is ethylene carbodiimide (ECDI).

7. The composition of claim 1, wherein the apoptotic signaling molecule is annexin-1, annexin-5, phosphatidyl serine, milk fat globule-EGF-factor 8 (MFG-E8), Fas-ligand or TNFalpha.

8. (canceled)

9. The composition of claim 1, wherein the antigenic peptide is fused to the apoptotic signaling molecule.

10. The composition of claim 1, wherein the carrier particle is a nanoparticle or microparticle.

11. The composition of claim 10, wherein the nanoparticle or microparticle is between 1 and 20 microns in diameter.

12. The composition of claim 10, wherein the nanoparticle or microparticle is biodegradable.

13-17. (canceled)

18. A method of reducing an antigen-specific immune response in a subject comprising administering to said subject a composition for induction of antigen-specific tolerance comprising a carrier particle attached thereto an apoptotic signaling molecule and an antigenic peptide, wherein said composition reduces an antigen-specific immune response in a subject.

19. The method of claim 18, wherein the antigenic peptide is an autoimmune antigen, a transplantation antigen or an allergen.

20. The method of claim 19, wherein the autoimmune antigen is one to which the subject mounts an immune response.

21. The method of claim 18, wherein the antigenic peptide is myelin basic protein, acetylcholine receptor, endogenous antigen, myelin oligodendrocyte glycoprotein, pancreatic beta-cell antigen, insulin, glutamic acid decarboxylase (GAD), collagen type 11, human carticlage gp39, fp130-RAPS, proteolipid protein, fibrillarin, small nucleolar protein, thyroid stimulating factor receptor, histones, glycoprotein gp70, pyruvate dehydrogenase dehyrolipoamide acetyltransferase (PCD-E2), hair follicle antigen or human tropomyosin isoform 5.

22. The method of claim 18, wherein the antigenic peptide is coupled to ECDI.

23. The method of claim 18, wherein the apoptotic signaling molecule is annexin-1, annexin-5, phosphatidyl serine, milk fat globule-EGF-factor 8 (MFG-E8), Fas-ligand or TNFalpha.

24-27. (canceled)

28. The method of claim 18, wherein the antigen-specific immune response is an autoimmune response, allergy, asthma, graft-versus-host reaction or graft rejection reaction.

29. The method of claim 18, wherein the composition is delivered orally, nasally, intravenously, intramuscularly, parenterally, ocularly or subcutaneously.

30-32. (canceled)

33. A kit for inducing antigen specific tolerance comprising:
(a) A carrier particle; and
(b) An antigenic peptide bound to the carrier particle.


SEQUENCE LISTING (3 sequences)

SEQ ID NO: 1 - protein, 13 aa, Artificial Sequence
  Description of Artificial Sequence Synthetic peptide
HSLGKWLGHPDKF

SEQ ID NO: 2 - protein, 17 aa, Artificial Sequence
  Description of Artificial Sequence Synthetic peptide
ISQAVHAAHAEINEAGR

SEQ ID NO: 3 - protein, 15 aa, Artificial Sequence
  Description of Artificial Sequence Synthetic peptide
GFKGVDAQGTLSKIF